(19) United States
(12) Patent Application Publication
(10) Pub. No.: US 2007/0281036 A1
(43) Pub. Date: Dec. 6, 2007

(54) System and method of delivering a desired material to a cell

(75) Inventors: Christopher C. Landry, Burlington, VT (US); Kai Cheng, Colchester, VT (US)

Correspondence Address: WOLF GREENFIELD & SACKS, P.C., 600 ATLANTIC AVENUE, BOSTON, MA, 02210-2206, US

(73) Assignee: University of Vermont and State Agricultural College, Burlington, VT (US)

(21) Appl. No.: 11/641,605

(22) Filed: Dec. 19, 2006

Related U.S. Application Data
(60) Provisional application No. 60/751,774, filed on Dec. 19, 2005.

(51) Int. Cl. A61K 9/14; A61K 31/17; A61K 31/282; A61K 31/4164; A61P 43/00; A61P 11/00; A61K 31/7028; A61K 47/02

(52) U.S. Cl. 424/497; 514/34; 514/398; 514/492; 514/589; 514/769

(57) ABSTRACT

The invention relates to compositions of mesoporous inorganic oxide particles and methods of use. The particles may be coated with a polyethylene glycol group. The compositions of the invention are useful for delivering therapeutic and diagnostic materials to an organism.

### RELATED APPLICATIONS

This application claims priority under 35 U.S.C. § 119(e) to U.S. Provisional Application Ser. No. 60/751,774, entitled “System and method for delivering a desired material to a cell” filed on Dec. 19, 2005, which is herein incorporated by reference in its entirety.

### FIELD OF THE INVENTION

The present invention relates generally to delivering biological agents to a tissue using mesoporous inorganic oxide spherical particles as well as related compositions thereof and methods of using the particles for therapeutic purposes such as treating lung disorders.

### BACKGROUND OF THE INVENTION

Porous silica is commonly used as a matrix material for chromatographic separations. With surface areas in the neighborhood of 300 meter(m)²/gram(g), commercially available chromatographic grade silicas possess a relatively high surface area. Mesoporous materials, which typically possess surface areas in excess of 1000 m²/g and even as high as 1600 m²/g, are commonly used as adsorbents, catalysts, and catalytic supports. With such high surface areas, these materials should provide superior separating ability as a chromatographic matrix in liquid chromatography (LC), flash liquid chromatography (FLC), and high performance liquid chromatography (HPLC). Mesoporous inorganic oxide particles differ from conventional porous inorganic oxides in that their surface areas are significantly larger than those of conventional porous inorganic oxides.

### SUMMARY OF THE INVENTION

The invention in some aspects relates to compositions of mesoporous inorganic oxide particles, particularly silicate particles and methods of using such particles to deliver biological agents.

Thus in one aspect the invention is a composition of a mesoporous inorganic oxide spherical particle coated with a polyethylene glycol group The mesoporous inorganic oxide spherical particle has pores loaded with a biological agent.

In other aspects the invention is a composition of a mesoporous inorganic oxide spherical particle having one or more pores, wherein the pores are loaded with a chemotherapeutic agent and wherein a linker is attached on one end to an external surface of the mesoporous inorganic oxide spherical particle and on another end to a tumor molecule binding agent.

In yet other aspects the invention is a composition of a mesoporous inorganic oxide spherical particle having one or more pores with a pore volume of greater than 0.75 cm³/g and an average pore diameter of greater than 37 Angstroms, wherein the pores are loaded with a biological agent.

In some embodiments the mesoporous inorganic oxide spherical particle is coated with an agent such as a polyethylene glycol group. The polyethylene glycol group may be tetraethylene glycol (TEG).

The mesoporous inorganic oxide spherical particle may or may not have a linker attached to an external surface. In some embodiments the linker is —Cₙ—SSpyNO₂. n is 1-20. The linker may be attached to a compound such as a protein or a tumor molecule binding agent. The tumor binding agent may be a protein. In some embodiments the protein is an antibody, a pleural tissue binding protein, a lung tissue binding protein, an epithelial cell binding protein, or a glycoprotein such as, for instance, CD4 or a fragment thereof.

In some embodiments the biological agent is a chemotherapeutic, such as, for instance, doxorubicin, carmustine, cisplatin, dacarbazine, LY294002, or PX866. In other embodiments the biological agent may be an anti-infective agent such as an anti-viral agent or an antibiotic. The anti-viral agent in some embodiments is an anti-HIV agent.

The mesoporous inorganic oxide spherical particle has a pore size that is useful for delivering biological agents to tissues. In some embodiments the pores of the particles have a pore volume of greater than 0.50 cm³/g, greater than 0.75 cm³/g, greater than 1.0 cm³/g, or greater than 1.3 cm³/g or any intervals there between. In other embodiments the particles have an average pore diameter of greater than 37 Angstroms, greater than 50 Angstroms, or between 2 and 200 nm.

The mesoporous inorganic oxide spherical particle may have a particle size between 0.5 and 10 μm in some embodiments.

The invention also relates to methods of delivering biological agents to tissue using mesoporous inorganic oxide spherical particles. In one aspect a method for delivering a biological agent to a tissue is provided. The method involves administering to a tissue of a subject a mesoporous inorganic oxide spherical particle coated with a polyethylene glycol group, wherein the mesoporous inorganic oxide spherical particle has pores loaded with a biological agent in an effective amount to deliver the biological agent to the tissue.

In yet another aspect the invention is a method for delivering a biological agent to a tissue, by administering to a tissue of a subject a mesoporous inorganic oxide spherical particle having one or more pores with a pore volume of greater than 0.75 cm³/g and an average pore diameter of greater than 37 Angstroms, wherein the pores are loaded with a biological agent in an effective amount to deliver the biological agent to the tissue.

In other aspects the invention is a method of treating lung or pleural disease in a subject by administering to a subject in need thereof a mesoporous inorganic oxide spherical particle coated with a polyethylene glycol group, wherein the mesoporous inorganic oxide spherical particle has pores loaded with a lung or pleural therapeutic agent in an effective amount to treat the disease.

In some embodiments the subject has a disease such as lung cancer, malignant mesothelioma, a pleural infection or melanoma.

The mesoporous inorganic oxide spherical particle may be administered to the subject using any known mode of delivery. In some embodiments the delivery mode involves administration intranasally, by an intrathoracic route, or by an intraperitoneal route.

In some embodiments the method is achieved using any of the mesoporous inorganic oxide spherical particles described above and herein.

Use of a composition or particle of the invention for delivering compounds to a tissue and treating lung or pleural disease is also provided as an aspect of the invention.

A method for manufacturing a medicament of a composition or particle of the invention for delivering compounds to a tissue and treating lung or pleural disease is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The FIGS. are illustrative only and are not required for enablement of the invention disclosed herein.

FIG. 1 is TEG facilitates cell uptake of APMS which are not cytotoxic to lung epithelial (C10) cells as measured by release of LDH. The effect of surface coatings on APMS-induced cytotoxicity was measured by the LDH assay (A), where 300 □M H₂O₂ was used as a positive control for LDH release. B. shows a dose response study using APMS coated with TEG where cell lysis was also measured by the LDH assay. Complete cell lysis was used as a positive control for LDH release. Results are expressed as Mean ±SEM from 3 experiments; *=p<0.01 when compared to control group at respective time point.

FIG. 2 is individual and clusters of APMS-TEG are internalized by MM cells. Panels in G show EDS analyses. Crosshairs on SEM images, as indicated by white arrows, indicate area probed. The corresponding elemental analysis shows the detection of APMS containing silica in upper panels whereas the lower panel shows no silica signal.

FIG. 3 is TEM images showing APMS-TEG (arrows) in the cytosol and intercellularly at 1 hr after their addition to MM cells.

FIG. 4 is a bar graph depicting the results of flow cytometry after incubation of MM cells at 4° C. or 37° C. and with or without selective inhibitors (pretreatment for 30 min before addition of APMS) were used to evaluate the percentage of cells positive for APMS uptake at 30 min (left panels) or 4 hrs (right panels). Mean ±SEM from at least 3 individual experiments. #=p<0.05; *=p<0.01 when compared to 37° C. group at respective time point.

FIG. 5 is a series of bar graphs depicting immunogenicity of APMS-TEG when injected IP or instilled IN in mice. Mean ±SEM of individual experiments performed on a minimum of at least 4 mice/group/time point.

FIG. 6 is APMS loaded with DOX is superior to DOX alone or added simultaneously with unloaded APMS in MM cell killing as assessed by the LDH assay. Arrows indicate approximate DOX responses at 65 nM concentrations.

FIG. 7 is more DOX is transferred to cells when it is preloaded into APMS than when it is administered alone. FIG. 7A, intracellular and extracellular amounts of DOX were measured by fluorescence HPLC after digestion and release of DOX from cellular material. Note the sudden decrease in the amount of intracellular DOX at 48 h due to cell death and lysis. FIG. 7B, a similar experiment was performed except that the DOX was added directly to the culture medium. Approximately half as much DOX was transferred to the cell in this experiment than the one shown in FIG. 7A.

FIG. 8 is absorption studies of calf thymus DNA (˜2000 base pairs) and a DNA plasmid for red fluorescent protein (RFP). FIG. 8A shows uptake studies using Mg⁺² as the counterion and several pore diameters. In FIG. 8B, adsorption of DNA plasmid by APMS containing Mg⁺² was studied. FIG. 8C shows the percent release of linear and plasmid DNA versus time.

FIG. 9 is knockdown of mek5 in MMil cell line with 4 siRNA constructs. QRT-PCR using SyberGreen. Gene expression is compared to the scramble control group.

FIG. 10 is phase contrast microscope images of cells exposed to water (control), 80 nM DOX, 160 nM DOX, 7.5×10⁶/cm² surface area dish APMS-TEG, 15×10⁶/cm² surface area dish APMS-TEG, APMS-DOX (80 nM), or APMS-DOX (160 nM) (FIG. 3A). LDH release measured to compare cytotoxicities (FIG. 3B-D).

FIG. 11 is APMS delivery of 80 nM DOX (APMS-DOX) decreases human mesothelioma cell (MM) viability. Panel A show a series of phase contrast microscopy images of MM cells exposed to either water (control), 80 nM DOX, APMS coated with tetraethylene glycol (APMS-TEG), or APMS-DOX, for varying amounts of time. Panel B shows cell viability as measured by MTS of either control treated MM cells, MM cells incubated with 80 nM DOX, APMS-TEG, APMS-DOX, or untreated MM cells.

FIG. 12 is APMS delivering 80 nM doxorubicin (APMS-DOX) does not increase phosphorylated H2AX (gH2AX) protein in human mesothelioma (MM) cells. Bar graph summarizing the percent of MM cells expressing gH2AX after treatment with either 80 nM DOX, APMS coated with tetraethylene glycol (APMS-TEG), APMS-DOX, for varying amounts of time

FIG. 13 is APMS delivering 80 nM doxorubicin (APMS-DOX80) may affect cell viability through an NF-kB-mediated mechanism. The effect of APMS and DOX on the activation of the cellular signaling molecules, ERK 1/2, AKT as determined using Western Blot Analysis, and a time course of phosphorylated AKT protein and total AKT protein expression were also determined using Western Blot Analysis. The bar graph shows a summary of densitometry analyses of at least two independent experiments.

### DETAILED DESCRIPTION

The invention is based in part on the discovery that compositions of mesoporous inorganic oxide particles, particularly silicate particles are useful for delivering biological agents to subjects. The properties of these particles enable effective in vivo delivery of large amounts of biological agents, while in some instances avoiding toxic effects associated with the agents themselves or other carriers.

Acid-prepared mesoporous spheres (APMS) are amorphous silica-based particles created synthetically. Unlike crystalline silica, amorphous silica is not associated with the development of lung disease and is non-toxic after administration via a number of routes in vivo or in vitro. Importantly, the APMS contain pores of various size dimensions that can allow them to be loaded with therapeutic drugs and other cargo. Both the diameter of the APMS and the pore size can be modified independently to deliver “cargo” of different solubility and molecular weights.

Nanoparticles, defined as particles of less than 0.1 μm in diameter, have been proposed in the treatment of a number of diseases. However, due to their small size, many of these particles can enter cell organelles and disrupt normal cell functions. Additionally, various particles in the nano-scale range also have systemic effects as they are dispersed by the systemic circulation and may also cross the blood-brain barrier. Thus, there are general concerns regarding potential toxic and systemic effects of nanoparticles after administration. To circumvent these problems, the invention involves respirable fine particles of greater than 0.1 μm in diameter for the delivery of agents, avoiding some toxic effects.

Mesoporous inorganic oxide particles differ from conventional porous inorganic oxides in that their surface areas are significantly larger than those of conventional porous inorganic oxides. For example, the surface area of the mesoporous inorganic oxide particles of the present invention are in excess of about 800 m²/g and, in some cases, in excess of 1200 m²/g. In comparison, well known inorganic oxide, conventional chromatographic grade silicas, generally have a surface area less than 500 m²/g, and commonly less than 300 m²/g.

Additionally, the pores may have a pore volume of greater than 0.75 cm³/g and an average pore diameter of greater than 37 Angstroms. In some embodiments the pores have a pore volume of greater than 1.0 cm³/g 1.3 cm³/g. In other embodiments the particles have an average pore diameter of greater than 50 Angstroms or between 2 and 200 nm, or between 0.5 and 10 μm.

The particles are loaded with biological agents and may be used in diagnostic or therapeutic methods. A diagnostic method as used herein refers to an in vivo method that does not involve treatment of a disease or condition but rather involves the detection or identification of a compound within the body. For instance the particles may be useful diagnostically with imaging agents such as those used in positron emission tomography (PET), computer assisted tomography (CAT), single photon emission computerized tomography, x-ray, fluoroscopy, and magnetic resonance imaging (MRI).

A therapeutic method may be any type of method involving delivery of a therapeutic agent to improve the outcome of a disease or disorder or prevent the onset or progression of a disease or disorder. Therapeutically the particles may be useful for treating conditions in which the biological agent may be useful for treatment or prophylaxis or for the alleviation of symptoms of a disease. Diseases or disorders include, for example, bacterial infections, viral infections, cancer, CNS disorders, cardiovascular disease and allergic diseases. Specific examples of conditions include diabetes, genetic disorders, pain, addiction, insomnia and sleep disorders, eating disorders, epilepsy, psychiatric disorders, Parkinson's disease, Alzheimer's disease, depression, stroke, rheumatic diseases, asthma, eczema, HIV and AIDS, gastric disorders and hypertension.

Thus, the particles may be useful in the treatment of a subject having or at risk of having cancer. A subject at risk of developing a cancer is one who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission.

A subject having a cancer is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to diagnose or treat diseases or conditions in non human subjects. For instance, cancer is one of the leading causes of death in companion animals (i.e., cats and dogs).

As used herein, the term treat, treated, or treating when used with respect to an disorder such as cancer refers to a prophylactic treatment which increases the resistance of a subject to development of the disease or, in other words, decreases the likelihood that the subject will develop the disease as well as a treatment after the subject has developed the disease in order to fight the disease or prevent the disease from becoming worse.

In some aspects of the invention the particles of the invention are useful for treating respiratory diseases such as airway or pleural diseases. For instance, it is believed that targeted drug delivery of the particles of the invention intranasally (IN) or intrapleurally (IP) would be non-toxic and beneficial in a number of airway and pleural diseases. Lung cancers, malignant mesotheliomas (MM), and fibrosis are devastating diseases with limited treatment strategies, in part due to poorly effective drug delivery to affected areas of lung. The inventors proposed that acid prepared mesoporous spheres (also referred to herein as APMS) of the invention might be effective vehicles for pulmonary chemotherapeutic drug delivery. To assess this, APMS, chemically modified with different surface molecules [lipid, a linker having a terminal amine group, a thiol group, or tetraethylene glycol (TEG)], were evaluated for uptake and cytotoxic effects following in vitro administration to murine alveolar epithelial Type II (C10) and human mesothelioma (MM) cells and after intrapleural (IP) or intranasal (IN) administration to C57/BL6 mice. The results of these studies are shown in the Examples section.

A series of surface modifications on the particles were first explored to increase efficiency of APMS uptake by cells and to elucidate possible mechanisms of cell internalization of APMS. In our experiments, APMS were synthesized and coated with different molecules including tetraethylene glycol (TEG), a short-chain polyethylene glycol. Since APMS-TEG were most avidly taken up by lung epithelial (C10) and human MM cells and were non-toxic to cells at high concentrations, the mechanisms of uptake F of APMS-TEG were characterized further. Unlike ultrafine or nanoparticles, APMS were not associated with the endoplasmic reticulum, mitochondria or endocytotic vesicles. In contrast to pathogenic particulates such as asbestos or silica, cell uptake of APMS-TEG did not involve encapsulation of particles by membranes into phagosomes nor their merging with lysosomes. Their ability to circumvent cellular digestion is a unique feature of APMS-TEG that should permit their delivery of “cargo” intracellularly without potential degradation after inhalation or IP administration. In the treatment of lung and pleural diseases, APMS-TEG should prove to be useful for delivering therapeutics such as chemotherapeutic drugs or molecular constructs to affected tissues.

Thus, the invention is useful in the treatment of mesothelioma. Mesothelioma is a disease in which cancer (malignant) cells are found in the sac lining the chest (the pleura), the lining of the abdominal cavity (the peritoneum) or the lining around the heart (the pericardium). Most patients now being diagnosed with malignant mesothelioma have been exposed to asbestos at some point. The term “mesothelioma” as used herein means in particular malignant mesothelioma, such as pleural mesothelioma, peritoneal mesothelioma, pericardial mesothelioma, epithelial mesothelioma, sarcomatous mesothelioma and biphasic mesothelioma. Early symptoms of the disease include shortness of breath, pain in the chest, or pain or swelling in the abdomen. In the diagnosis of mesothelioma an x-ray or CT scan of the chest or abdomen is often performed. If further examination is warranted, thoracoscopy, peritoneoscopy or biopsy can be conducted. Pathology, the scientific study of cells, tissue, or fluid taken from the body, is an integral part of a mesothelioma diagnosis.

The particles of the invention are also useful in the treatment of melanoma. The term “melanoma” includes but is not limited to melanomas, metastatic melanomas, melanomas derived from either melanocytes or melanocyte related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma and familial atypical mole and melanoma (FAM-M) syndrome, for example.

Metastatic melanoma may be treated with chemotherapeutic agents including for instance BELD (bleomycin, vindesine, lomustine, and deacarbazine), BOLD (bleomycin, vincristine, lomustine, dacarbazine;); DD (dacarbazine, actinomycin), or POC (procarbazine, vincristine, lomustin) among others. Other suitable chemotherapeutic regimens may also be utilized.

In general, melanomas may result from chromosomal abnormalities, degenerative growth and development disorders, mitogenic agents, ultraviolet radiation (UV), viral infections, inappropriate tissue expression of a gene, alterations in expression of a gene or carcinogenic agents. Melanoma is currently diagnosed by assessing risk factors and by performing biopsies. Risk factors for melanoma are a family history of melanoma, the presence of dysplastic nevi, patient history of melanoma, weakened immune system, many ordinary nevi, exposure levels to ultraviolet radiation, exposure to severe sunburns especially as a child or teenager, and fair skin. In a biopsy, a pathologist typically examines the biopsied tissue under a microscope to identify cancer cells. Depending upon the thickness of a tumor, if one exists, a physician may order chest x-ray, blood tests, liver scans, bone scans, and brain scans to determine whether the cancer spread to other tissues.

The particles have a spherical shape that is easily recognized by microscopy and the large internal pore area may be used to store biomolecules (i.e. DNA, RNA) or pharmaceuticals (i.e. anticancer drugs). Additionally, the outer surface of APMS can be modified with various molecules for affinity binding. We have developed protection/deprotection methods by which the linkers can be exclusively attached to the external bead surface. Thus, peptides or proteins can be covalently attached to the external surface of the bead, leaving the internal pore structure available to carry exchangeable molecules after complete deprotection. As described in the Examples particles having a CD4 group linked to the surface have been generated. The CD4-gp120 interaction has been studied for this purpose, since the peptide sequences on each glycoprotein responsible for the actual binding have been determined. The modifications of CD4 fragment and recombinant CD4 protein provided a convenient and efficient way for immobilization to the APMS surface without loss of their affinity for gp120 (Recombinant HIV-1 gp120 (LAV IIIB)). Both a short 17-peptide CD4 fragment, which is specific for affinity binding, and whole CD4 glycoprotein were able to recognize and bind gp120 when bound to the surface of APMS. In several experiments, gpl20 was completely removed from the solution. Such molecules may be useful in targeting particles to HIV. The protection/deprotection strategy provided a convenient and efficient way by which peptides or proteins could be attached to the external surface of a mesoporous silica particle without the blockage of the pore entrances.

Thus, in some embodiments the mesoporous inorganic oxide spherical particle has a linker attached to an external surface. A linker, as used herein, is a molecule that is capable of being attached to the particles directly or indirectly. Another molecule can also be attached to the linker. Linkers useful according to the invention include but are not limited to amine, methylamine, carboxylic acid, maleimide-succinimide, maleimide-hydrazine linkers, and —Cₙ—SSpyNO₂.

The linker may be attached to a protein or other molecule, for instance, for the purpose of targeting the particle to a particular tissue or cell. In some embodiments the protein is a glycoprotein, an antibody, a pleural tissue binding protein, a lung tissue binding protein, an epithelial cell binding protein, or a tumor molecule binding agent.

The APMS are spherical. A particle is considered spherical if it displays a spheroidal shape, whether free standing or attached to other particles. The spherical quality of a particle is measured using scanning electron microscopy (SEM). A spherical particle provides greater functionality than a non-spherical particle. Spherical particles pack together, for example in a chromatography column, such that there is always some empty space between them. In chromatography it is essential that some space exist between the column particles such that the sample molecules can flow around the column particles. Spherical particles also have the advantage of being more readily recognized by microscopic techniques, such as fluorescence microscopy and electron microscopy, which have difficulty distinguishing non-spherical particles from one another.

The particles may be synthesized by methods such as those described in co-pending U.S. Ser. No. 11/292178. Synthesis of APMS is typically accomplished by polymerizing an inorganic alkoxide (tetraethoxysilane, TEOS) in the presence of a surfactant (cetyltrimethylammonium bromide, CTAB). For instance, synthesis may be accomplished using an acidic aqueous reaction procedure over a shorter period of time than is used for synthesis for similar particles.

The temperature required during synthesis over the shorter periods of time to achieve a high percentage of spherical particles is lower than that of known processes. A lower temperature, preferably a temperature below the boiling point of water (100° C.), provides for more efficient and affordable scaling of the synthesis to commercial scales. The methods utilize a heating step that heats the reaction mixture at a temperature of about 50° C. to about 230° C.

The method of preparing a mesoporous inorganic oxide spherical particles involves a reaction mixture having a source of inorganic oxide and being capable of forming a mesoporous inorganic oxide sphere. The reaction mixture is heated for a selected time and organic material is removed from the resulting product to form a mesoporous inorganic oxide spherical particle having a desirably large pore volume. The methods may also include other constituents. Other constituents may include, but are not limited to, a source of fluoride, an alcohol, a proton donor, a surfactant, and water. The reaction mixture may also include a metal salt. The constituents of a reaction mixture may be mixed (e.g., by stirring) for a selected time prior to the mixture being heated.

A reaction mixture may include, for instance, an inorganic oxide, a source of fluoride, an alcohol, a proton donor, a surfactant, and water. In one example, a reaction mixture according to this embodiment may include TEOS, sodium fluoride, ethanol, hydrochloric acid, CTAB and water.

A source of inorganic oxide may include any material that is a source of silicate. In one aspect, a source of silicate can include a compound having a formula Si(OR¹)(OR²)(OR³)(OR⁴) where Si is silicon, O is oxygen, and ¹, R², R³, and R⁴ are alkyl chains having 1 to 4 carbon atoms. Examples of sources of silicate include, but are not limited to, tetraethoxysilane, tetramethoxysilane, tetrapropoxysilane, and any combinations thereof. In one embodiment of the present invention, the source of inorganic oxide is tetraethoxysilane (also known as tetraethyl orthosilicate or TEOS) sold by Sigma-Aldrich.

In one example, an inorganic oxide may be present in the reaction mixture in an amount from about 0.017 mole (mol) % to about 1.6 mol %. In another example, an inorganic oxide may be present in the reaction mixture of the present invention in an amount from about 0.3 mol % to about 1.2 mol %. In yet another example, an inorganic oxide may be present in the reaction mixture of the present invention in an amount from about 0.6 mol % to about 0.8 mol %. In still another example, an inorganic oxide may be present in the reaction mixture of the present invention in an amount of about 0.6 mol %.

The fluoride may be a salt that includes a fluoride ion. Examples of suitable sources of fluoride include, but are not limited to, sodium fluoride, potassium fluoride, ammonium fluoride, other fluoride salts, and any combinations thereof. In one embodiment of the present invention, the source of fluoride is a 0.5 Molar (M) solution of sodium fluoride prepared from sodium fluoride sold by Sigma-Aldrich.

The alcohol in the reaction may be a water-miscible alcohol. Examples of water-miscible alcohols include, but are not limited to, ethanol, methanol, n-propanol, isopropanol, and any combinations thereof. In one example, an alcohol includes 200 proof ethanol sold by AAPER Alcohol and Chemical Co.

A proton donor may include an acid. Examples of acids suitable for use as a proton donor include, but are not limited to, HCl, HBr, HI, HNO.sub.3, H.sub.2SO.sub.4. In one example, a proton donor includes a concentrated (37.2 wt. %) solution of hydrochloric acid sold by Fischer Scientific.

In one embodiment of the present invention, a surfactant includes a cationic surfactant. In another embodiment of the present invention, a surfactant includes a cationic ammonium having a formula R₁R₂R₃R₄N⁺X⁻, where R₁, R₂ and R₃ are alkyl chains consisting of 1 to 6 carbon atoms, R₄ is an alkyl chain consisting of 12 to 24 carbon atoms and X⁻ represents a counterion to said surfactant, said counterion selected from the group consisting of Cl⁻, Br⁻, I⁻ and OH⁻. In yet another embodiment of the present invention, a surfactant includes a tri-block copolymer EOₓPO_{y}EOₓ, where EO is polyethylene oxide, PO is polypropylene oxide and x ranges from 5 to 106, y ranges from 30 to 85 and z ranges from 5 to 106. In still yet another embodiment of the present invention, a surfactant includes a salt having a trialkylammonium cation and a halide anion. In a further embodiment of the present invention, multiple surfactants can be used. Using multiple surfactants is commercially advantageous in that various pore diameters and physical properties are introduced into the material in a single process.

Examples of suitable surfactants include, but are not limited to, cetyltrimethylammonium bromide, cetyltrimethylammonium chloride, tetradecyltrimethylammonium bromide, lauryltrimethylammonium bromide, lauryltrimethylammonium chloride, tetradecyltrimethylammonium chloride, poly(ethylene glycol)-block-poly(propylene glycol)-block-poly(ethylene glycol), and any combinations thereof. In one example, a surfactant includes cetyltrimethylammonium bromide (CTAB) sold by Sigma-Aldrich.

In one example, a surfactant is present in the reaction mixture of the present invention in an amount from about 0.01 mol % to about the limit of solubility. In another example, a surfactant is present in the reaction mixture of the present invention in an amount from about 0.119 mol % to about 0.26 mol %. In yet another example, a surfactant is present in the reaction mixture of the present invention in an amount of about 0.16 mol %.

The constituents of a reaction mixture are combined and mixed until chemically homogenous. In one example, this combination and mixing can be done at room temperature. In another example, the mixing can be done at a temperature of about 25° C. to about 35° C. The mixing may be accomplished by stirring, by sonication involving use of a sonication horn of the type sold by Heat Systems-Ultrasonics, Inc. of Farmingdale, N.Y. (one model of sonication horn operating at a frequency and maximum power, respectively, of 20,000 kHz and 475 watts), or by any other technique or techniques yielding a chemically homogenous mixture. After the addition of all reaction mixture constituents, the mixing is continued until the reaction mixture is sufficiently polymerized such that a mesostructured inorganic oxide sphere may be formed upon heating the reaction mixture. In one example, sufficient polymerization is indicated by the reaction mixture turning opaque. As used in this context, an “opaque” mixture means a mixture having a transparent to white color and containing a suspension of very small particles that cannot be captured by Buchner filtration on VWR qualitative filter paper grade 413. Typical times for mixing include about 15 seconds to about 2.5 hours, depending on the composition of the reaction mixture. In another example, the time of mixing is about 25 sec. to about 360 sec. While it is typically advantageous to achieve chemical homogeneity as quickly as possible, in some cases it may be desirable to extend the mixing period. This can be achieved by reducing either the acid and/or fluoride concentrations and/or increasing the ethanol concentration in the reaction mixture.

The reaction mixture is then heated at a temperature, time, and pressure sufficient to form one or more mesostructured inorganic oxide spheres. A mesostructured inorganic oxide sphere includes inorganic material, for example inorganic oxide, and organic material, for example surfactant, intimately combined as a composite particle. The heating step can take place in any vessel capable of withstanding the selected temperature, time, and pressure. One example of a vessel suitable for the heating step is a Teflon bottle. Another example of a vessel suitable for the heating step is a stainless steel autoclave, such as model 4748 and model 4749 t-lined stainless steel autoclaves sold by Parr Instruments Co. of Moline, Ill.

The reaction mixture is heated during this step at a temperature sufficient to produce the mesostructured inorganic oxide sphere. In one example, the reaction mixture is heated at a temperature from about 50° C. to about 230° C. In another example, the reaction mixture is heated at a temperature from about 70° C. to about 200° C. In yet another example, the reaction mixture is heated at a temperature from about 90° C. to about 150° C. In still another example, the reaction mixture is heated at a temperature below about 100° C. In still yet another example, the reaction mixture is heated at a temperature of about 70°.

The heating step occurs for a period of time sufficient to produce the mesostructured inorganic oxide sphere at the selected temperature. Lower temperatures typically require longer time for the same reaction mixture components. In one example, the reaction mixture is heated for not more than 120 minutes. In another example, the reaction mixture may be heated for a time that may range from about 10 minutes to about 80 minutes. In yet another example, the reaction mixture may be heated for a time that may range from about 20 minutes to about 60 minutes. In still another example, the reaction mixture may be heated for about 40 minutes.

The resulting mesostructured inorganic oxide sphere can be separated from any remaining reaction mixture by a conventional technique, such as filtration. The filtered mesostructured inorganic oxide sphere can be dried using a conventional technique, such as vacuum filtration. The filtration and drying steps may be combined or separate steps. In one example, the drying of a mesostructured inorganic oxide sphere is performed at about room temperature. In another example the drying can occur at any temperature as long as it is not high enough to cause decomposition of the surfactant.

Organic material, such as surfactant, is removed from a mesostructured inorganic oxide sphere to produce a mesoporous inorganic oxide sphere. Examples of suitable F techniques for removing the organic material include, but are not limited to, burn away of the organic material away with such a technique as calcining, wash-out of the organic material, ion exchange, and any combination thereof. In one example, the organic material is removed by heating the material to a temperature in the range of about 400° C. to about 600° C. with a temperature ramp of about 0.2° C./minute to about 5° C./minute, preferably no more than 2° C./minute, and then maintaining the material at such temperature for at least about 6 hours. In another example, the organic material is removed in a two-step process where the mesostructured inorganic oxide sphere is heated at a temperature ramp of about 2° C./minute to a temperature of about 450° C., where it is maintained for about 4 hours. Then, the temperature is elevated at a temperature ramp of about 10° C./minute to 550° C., where it is maintained for about 8 hours. In yet another example, the organic material is removed by ion exchange using dilute HCl dissolved in ethanol.

Unlike particles that are a thin silica shell formed around a single large void (often formed by polymerizing a spherical silica shell around an oil droplet) a mesoporous inorganic oxide sphere produced by a method of the present invention is not hollow, but has a mesoporous region that continues throughout the interior of the particle.

Mesoporous inorganic oxide spheres produced in accordance with the present invention have a particle diameter that is advantageously small. In one example, the diameter of mesoporous inorganic oxide spheres produced by a method of the present invention range from about 0.1 μm to about 8 μm. In another example, the diameter of mesoporous inorganic oxide spheres produced by a method of the present invention range from about 1 μm to about 3 μm.

A mesoporous inorganic oxide sphere produced in accordance with the present invention has a large area. Larger surface area is particularly important in capturing therapeutic agents required in high concentrations, where the larger the surface area, the higher the concentration that can be achieved in vivo. In one example, mesoporous inorganic oxide spheres produced in accordance with the present invention have a surface area from about 400 m²/g to about 1200 m²/g. In another example, mesoporous inorganic oxide spheres produced in accordance with the present invention have a surface area from about 800 m²/g to about 1000 m²/g.

F Biological agents include both diagnostics and therapeutics. Thus, any of a variety of diagnostic agents can be incorporated within the particles, which can locally or systemically deliver the incorporated agents following administration to a patient. Any biocompatible or pharmacologically acceptable gas or dye can be incorporated into the particles or trapped in the pores of the particles using technology known to those skilled in the art.

Any of a variety of therapeutic or prophylactic agents also can be incorporated within the particles, or used to prepare particles. Examples include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, plasmids, vectors, antisense molecules which bind to complementary DNA to inhibit transcription, siRNA, shRNA, and ribozymes.

In general, the biological agents include, but are not limited to: an adrenergic agent; adrenocortical steroid; adrenocortical suppressant; amine deterrent; aldosterone antagonist; amino acid; amnnonia detoxicant; anabolic; analeptic; analgesic; androgen; anesthesia; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal;. anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemic; antihyperlipoproteinemic; antihypertensive; antihypotensive; anti-infective; anti-infective, topical; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; inhibitor; keratolytic; LNRH agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; scabicide; sclerosing agent; sedative; sedative-hypnotic; selective adenosine Al antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; agent for treatment of amyotrophic lateral sclerosis; agent for treatment of cerebral ischemia; agent for treatment of Paget's disease; agent for treatment of unstable angina; uricosuric; vasoconstrictor; vasodilator; vulnerary; wound healing agent; and xanthine oxidase inhibitor.

Biological agents also include immunological agents such as allergens (e.g., cat dander, birch pollen, house dust, mite, grass pollen, etc.) and antigens from pathogens such as viruses, bacteria, fungi and parasites. These antigens may be in the form of whole inactivated organisms, peptides, proteins, glycoproteins, carbohydrates or combinations thereof. Specific examples of pharmacological or immunological agents that fall within the above-mentioned categories and that have been approved for human use may be found in the published literature.

In certain instances, the biological agent is an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Examples of anti-parasitic agents, also referred to as parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflomithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents include, but are not limited to, basiunginWECB, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine.

In other instances, the biological agent is a chemotherapeutic agent. Chemotherapeutic agents may be selected from the group consisting of methotrexate, vincristine, adriamycin, cisplatin, taxol, paclitaxel, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, dacarbazine, LY294002, PX866, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD001, ISI641, ODN 698, TA 2516/Marmistat, BB2516/Marmistat, CDP 845, D2163, PD183805, DX8951f, Lemonal DP 2202, FK 317, Picibanil/OK-432, AD 32[Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

The immunotherapeutic agent may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA, but it is not so limited.

Other anti-neoplastic compounds include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A₂; bleomycin B₂; bropirimine; budotitane; buthionine sulfoximine; calcipotriol; calphostin C; camptothecin derivatives (e.g., 10-hydroxy- camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflomithine; elemene; emitefur; epirubicin; epothilones (A, R═H; B, R═Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl amine; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Another category of therapeutic agents for use in the present invention is antipsychotic agents. Antipsychotic agents include lorazepam; chlordiazepoxide; clorazepate; diazepam; alprazolam; hydroxyzine; buspirone; venlafaxine; mephobarbital; meprobamate; doxepin; perphenazine; hydroxyzine pamoate; venlafaxine; mirtazapine; nefazodone; bupropion; phenelzine; tranylcypromine; citalopram; paraxefine; sertraline; amitrptyline; protriptyline; divalproex; clonazepam; clozapine; haloperidol; loxapine; molindone; thiothixene; pimozide; risperidone; quefiapine; thiothixen; olanzapine; quetiapine; prochlorperazine; mesoridazin; trifluoperazine; chlorpromazine; perphenazine; and fluvoxamine.

Non-steroidal anti-inflammatory agents (NSAIDS) may be incorporated in the particles. NSAIDS, such as propionic acid derivatives, acetic acid, fenamic acid derivatives, biphenylcarboxylic acid derivatives, oxicams, include but are not limited to aspirin, acetaminophen, ibuprofen, naproxen, benoxaprofen, flurbiprofen, fenbufen, ketoprofen, indoprofen, pirprofen, carporfen, and bucloxic acid and the like. In a preferred embodiment, the compositions of the invention comprise a therapeutically or diagnostically effective amount of the biological agent. Concentrations of the biological agents in the compositions of the invention are in the range of about 10 to about 100 percent (wt/wt). A more preferred range of concentrations is from about 25 to about 100 percent, and even more preferred concentrations are from about 40 to about 100 percent. In particular, an amount selected from the group consisting of about 10, about 25, about 40; about 99 and about 100 percent (wt/wt) is preferred.

As used herein, the term “therapeutically effective amount” means an amount of the composition of the invention that is sufficient to show a meaningful patient benefit, i.e., healing or amelioration of the disease or disorder, a reduction in one or more symptoms, or an increase in rate of healing of such diseases or disorders. Therapeutic efficacy and toxicity of the compositions may be determined by standard pharmaceutical, pharmacological, and toxicological procedures in cell cultures or experimental animals. For example, numerous methods of determining ED₅₀ (the dose therapeutically effective in 50 percent of the population) and LD₅₀ (the dose lethal of 50 percent of the population) exist. The dose ratio between therapeutic and toxic effects is the therapeutic index, and it can be expressed as the ratio ED₅₀I/LD₅₀. Compositions exhibiting high therapeutic indices are preferred. The data obtained from cell culture assays or animal studies may be used in formulating a range of dosages for human use. The dosage is preferably within a range of concentrations that includes the ED₅₀ with little or no toxicity, and may vary within this range depending on the dosage form employed, the type of disorder being treated, tolerance of side effects, sensitivity of the patient, and the route of administration.

Combined with the teachings provided herein, by choosing among the various h active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. One of ordinary skill in the art can empirically determine the effective amount of a particular therapeutic agent in mesoporous particles without necessitating undue experimentation.

Subject doses of the compounds described herein for mucosal or local delivery typically range from about 0.1 μg to 10 mg per administration, which depending on the application could be given daily, weekly, or monthly and any other amount of time therebetween.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the mesoporous particles can be administered to a subject by any mode that delivers the particle to the desired surface. Administering the pharmaceutical composition of the present invention may be accomplished by any means known to the skilled artisan. Preferred routes of administration include but are not limited to oral, parenteral, intramuscular, intranasal, sublingual, intrathoracic, intraperitoneal, intratracheal, inhalation, ocular, vaginal, and rectal.

The particles may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The particles may be administered alone or in any appropriate pharmaceutical carrier, such as a liquid, for example saline, or a powder, for administration in vivo. They can be co-delivered with larger carrier particles.

The mesoporous silica particles of the invention may be systemically administered in combination with a pharmaceutically acceptable vehicle such as an inert diluent or an assimilable edible carrier. They may be enclosed in hard or soft shell gelatin capsules or compressed into tablets. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed.

The mesoporous silica particles of the invention may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For topical administration, the mesoporous silica particles of the invention will generally be administered as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Thickeners such as synthetic polymers, fatty acids, fatty acid salts and esters, fatty alcohols, modified celluloses or modified mineral materials can also be employed with liquid carriers to lform spreadable pastes, gels, ointments, soaps, and the like, for application directly to the skin of the user.

The compositions of the inventions may include a physiologically or pharmaceutically acceptable carrier, excipient, or stabilizer mixed with the particles. The term “pharmaceutically acceptable” means a non-toxic material that does not interfere with the effectiveness of the biological activity of the active ingredients. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Particles may also be suspended in non-viscous fluids and nebulized or atomized for administration of the dosage form to nasal membranes. Particles may also be delivered parenterally by either intravenous, subcutaneous, intramuscular, intrathecal, intravitreal or intradermal routes as sterile suspensions in isotonic fluids.

Finally, particles may be nebulized and delivered as dry powders in metered-dose inhalers for purposes of inhalation delivery. For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of for use in an inhaler or insufflator may be formulated containing the microparticle and optionally a suitable base such as lactose or starch. Those of skill in the art can readily determine the various parameters and conditions for producing aerosols without resort to undue experimentation. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Techniques for preparing aerosol delivery systems are well known to those of skill in the art. Generally, such systems should utilize components which will not significantly impair the biological properties of the agent in the nanoparticle or microparticle (see, for example, Sciarra and Cutie, “Aerosols,” in Remington's Pharmaceutical Sciences, 18th edition, 1990, pp. 1694-1712; incorporated by reference).

Particles when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

Materials and Methods:

Cells and Reagents:

Murine alveolar epithelial Type II cells (C10) (23) were maintained in CMRL 1066 (P139-500, Biosource, Rockville, Md.) supplemented with 10% fetal bovine serum (FBS) (35-010-CV, Mediatech, Herndon, Va.), 2 mM L-glutamine (25030-156, GIBCO, Invitrogen, Carlsbad, Calif.), and penicillin-streptomycin (50 U/ml penicillin G, 50 μg/ml streptomycin sulfate) (15140-122, GIBCO). Human mesothelioma cells (MM) (obtained from Dr. Luciano Mutti, Maugeri Foundation, Pavia, Italy) were maintained in DMEM/F12 50/50 medium (10-092-CV, Mediatech) supplemented with 10% FBS, 0.1 μg/ml hydrocortisone (H-0135, Sigma, St. Louis, Mo.), 2.5 μg/ml insulin, 2.5 μg/ml transferrin, 2.5 ng/ml sodium selenite (I-1884, Sigma) and penicillin-streptomycin (50 U/ml penicillin G, 50 μg/ml streptomycin sulfate) (15140-122, GICO). Unless otherwise specified, reagents were purchased from Sigma.

Synthesis of APMS:

Cetyltrimethylammonium bromide (CTAB) (4.9 mM) was suspended in 53.75 ml of an ethanol and distilled water mixture (at a 1:2.8 ratio). Concentrated hydrochloric acid (HCl, 3.7 ml) was added, and the mixture stirred until fully dissolved. Tetraethoxysilane (TEOS, 4.29 ml) was then added with stirring for 5 min, followed by addition of 4.67 ml of 0.5 M sodium fluoride (NaF, in water), the mixture heated at 373 K for 40 min, and the resulting white precipitate captured by filtration, washed 1× with distilled H₂O, and air dried. The synthesized APMS were placed under high vacuum to remove the remaining moisture.

Synthesis of APMS-NHMe, APMS-NH₂, and APMS-SH:

To modify their surface, APMS were reacted with an organosilane containing the target functionality. As an example, to synthesize secondary amine-terminated particles, APMS (1 g) and N-methyl-propylaminetrimethoxysilane (2 mM) were added to hexane (20 ml), and the mixture was heated at 333 K for 2 hrs. After cooling to room temperature, the solid was filtered, washed with hexane and ethanol, and dried at 373 K in air. To remove the surfactant (CTAB), 1 g of amine-modified APMS with N-methyl-propylamine functionality (APMS-NHMe) was refluxed twice for 6 hrs in ethanol followed by extensive washes with ethanol. The resulting APMS-NHMe then was dried under high vacuum. To prepare APMS-NH₂ and APMS-SH, identical procedures were followed except that the amine was replaced with aminopropyltrimethoxysilane and mercaptopropyltrimethoxysilane, respectively.

Synthesis of APMS-Lipid and Fluorescently Labeled APMS:

To synthesize APMS with a lipid-modified surface, APMS-NH₂ (0.300 g) was suspended in a solution of oleic acid (0.349 g) in N,N′-dimethylformamide. Standard peptide bond-forming methodology (24) was used to attach the acid to the surface, and after stirring at room temperature for 12 hrs, the resulting material was captured by filtration, washed and dried in air, and stored under vacuum. APMS could easily be covalently labeled with appropriate fluorescent dyes, such as Alexa-488 succinimide ester (Molecular Probes, Eugene, Oreg.), by forming peptide bonds.

Synthesis of APMS-TEG:

A half gram of APMS-NHMe was suspended in 10 ml of anhydrous ethanol containing mono-tosylated tetraethylene glycol (Ts-TEG) (0.5 mM). The mixture was then refluxed for 6 hrs. The resulting APMS-TEG was captured by filtration, washed with ethanol, and dried in air. The APMS-TEG were placed under high vacuum to remove the remaining solvent.

Synthesis of APMS-TEG Preloaded with Plasmid:

2 mg of APMS-TEG modified with Alexa-633 was combined with 1 ml of 0.2 M MgCl₂, sonicated for 10 min, and incubated at room temperature overnight. The supernatant was decanted, and APMS were dried in a vacuum. 1 mg of the APMS were then combined with 0.2 ml PBS (pH 7.2) containing 10 μg of the plasmid, pCMV-DsRed-Express (Clontech Laboratories Inc., Mountain View, Calif.), and sonicated for 30 min. Samples were then centrifuged, APMS were washed 2 times in PBS, and samples were dried in a vacuum.

Treatment of Cells with APMS:

Cells were plated and grown to 70-80% confluence at 37° C. in complete medium. Medium was aspirated and replaced with maintenance medium containing 0.5% FBS, and incubated for 24 hrs. APMS were then resuspended in medium containing 0.5% FBS serum at a concentration of approximately 6×10⁷ APMS per 100 μl, mixed well, and sonicated 5 times for 2 sec, to disperse any clumps. 50 μl were then added to cells at a final density of 7.5×10⁶/cm² surface area dish (i.e. ˜185 particles/cell).

LDH Measurement for Measurement of Cell Damage in vitro:

Cells were treated with APMS for various amounts of time, and lytic cell damage was measured by determining levels of LDH released into the medium using Cytotox 96 kit (Promega, Madison, Wis.), as per manufacturer's recommendations.

Sample Preparation for Scanning Electron Microscopy (SEM) and Transmission Electron Microscopy (TEM):

Cells were grown on Thermanox plastic coverslips (Nalge Nunc International, Naperville, Ill.) in 12-well plates as described above, and treated with APMS for various amounts of time. Coverslips were washed 2 times for 5 min with 0.1 M Millonig's phosphate buffer (pH 7.2), then fixed in 1:1 H₂O dilution of Karnovsky's fixative (2.5% glutaraldehyde, 1% paraformaldehyde) at 4° C. for 45 min. Samples were then washed with Millonig's phosphate buffer (pH 7.2), and post-fixed in osmium tetroxide (OsO₄) at 4° C. for 30-45 min. Samples were then dehydrated in graded ethanols, from 35% to 100%.

Scanning Electron Microscopy (SEM):

Samples prepared as described above were critical point dried using liquid CO₂ as the transition fluid in a Samdri PVT-3B critical point dryer (Tousimis Research Corporation, Rockville, Md.). Specimens were mounted on aluminum specimen stubs using conductive graphite paint and allowed to dry, and were sputter-coated for 4-5 min with gold and palladium in a Polaron sputter coater (Model 5100, Quorum Technologies, Guelph, Ontario, Canada). Specimens were then examined with a JSM 6060 scanning electron microscope (JEOL USA, Inc., Peabody, Mass.).

Transmission Electron Microscopy (TEM) and Elemental Analysis:

Samples were prepared as described above and were infiltrated with Spurr's resin according to the following schedule: (100% ethanol:Spurr's resin) 3:1 for 30 min; 1:1 for 30 min; 1:3 for 30 min; and 100% Spurr's resin for 30 min. Flat embedding molds were filled with Spurr's resin, and coverslips were placed onto the surface of the resin, cell side down. Resin was then polymerized overnight at 70° C. Polymerized blocks were plunged into liquid nitrogen to facilitate peeling of the coverslips from the resin block, and the resin blocks were cut into pieces and remounted onto blank blocks for sectioning.

Semi-thin sections (1 ρm) were cut using glass knives on a Reichert Ultracut microtome, stained with methylene blue—azure II, and evaluated for areas of cells. Ultra-thin sections (60-80 nm) were cut with a diamond knife, retrieved onto 150 mesh copper grids, contrasted with uranyl acetate (2% in 50% ethanol) and lead citrate, and examined with a JEM 1210 TEM (JEOL USA, Inc.) operating at 60 kV.

Energy Dispersive Spectroscopy (EDS):

Grids from TEM samples were analyzed, or portions of coverslips processed for SEM were cut after critical point drying and fixed onto copper bulk holders with conductive tape. They were sputter-coated with gold and palladium as described above. Cells were imaged, and EDS was performed on samples by STEM with a JEM 1210 transmission electron microscope (JEOL USA, Inc.), equipped with an energy dispersive X-ray spectrometer and IMIX sqftware, version 7, (hardware and software from Princeton Gamma-Tech, Princeton, N.J.). Accelerating voltage on the JEOL was 20 kV. A spot analysis of APMS was performed with the IMIX collecting at 0-20 keV.

Confocal Scanning Laser Microscopy (CSLM):

Cells were grown on glass-bottom culture dishes (P35G-1.5-14-3, MatTek Corporation, Ashland, Mass.) and treated with APMS as described above. Cells were subsequently incubated with the nucleic acid dye, Hoechst 33342 (H3570, Molecular Probes, Invitrogen), for 15 min. Medium was then removed and replaced with 1 ml ice cold Ca⁺⁺ and Mg⁺⁺-free Hank's Balanced Salt Solution without Phenol Red, containing 5 μg/ml membrane dye FM4-64 (T3166, Molecular Probes, Invitrogen). Samples were kept on ice and immediately viewed on a Zeiss LSM 510 META confocal scanning laser microscope (Carl Zeiss Microimaging, Thornwood, N.Y.).

Determination of Mechanisms of APMS Uptake by Flow Cytometry:

Cells were exposed for 30 min to chemical inhibitors of specific uptake mechanisms: chlorpromazine (5 ρg/ml), cytochalasin D (1 μM), or filipin (6.25 μg/ml), i.e. at effective concentrations as reported in the literature (25-29), or incubated at 4° C. for 30 min prior to addition of APMS for various amounts of time. Hoechst 33342 nucleic acid dye (16.2 μM) (Molecular Probes, Invitrogen) was added 15 min prior to harvesting cells. Cells were washed once in PBS and removed from dishes using Accutase cell detachment solution (Innovative Cell Technologies, Inc. San Diego, Calif.), pelleted by centrifugation, resuspended in calcium and magnesium-free phosphate buffered saline (PBS), and kept on ice until analyzed. Samples were analyzed using a BD LSRII flow cytometer (BD Biosciences, San Jose, Calif.) equipped with a Sapphire 488 (Coherent, Santa Clara, Calif.) laser which emits at 488 nm to excite the Alexa 488 dye, and a solid state Xcite (Lightwave) which emits at 355 nm to excite the Hoechst 333342 nucleic acid dye (Molecular Probes, Invitrogen). Data analysis was performed at the time of acquisition using Flow Jo (Tree Star, Inc., Ashland, Oreg.). This software package is an experiment-based flow cytometry data analysis package designed for multi-color research.

To verify that the gating used in determining cell populations was accurate, FACS analysis was performed on different populations. Populations of cells/APMS were selected based on their locations on histograms, and were sorted, collected and mounted on glass slides using cytocentriftigation. Cell sorting was accomplished using a BD FACSARIA (BD Biosciences) equipped with the following lasers: a Sapphire 488 (Coherent) which emits at 488 nm to excite the Alexa 488 dye, and an IFlex 2000-P1-405 (Point Source, Southampton, UK) which emits at 407 nm to excite the Hoechst 33342 nucleic acid dye. The fluorescent signals from Alexa 488 were detected using 530/30 BP and 505 LP filters. Hoechst staining was detected using 440/40 BP filter. Identification and quantification of populations on cytospins were performed manually with phase contrast and fluorescence microscopy using an Olympus BX50 microscope (Olympus America, Lake Success, N.Y.).

Delivery of Functional Plasmid Using APMS.

C10 cells were grown on glass coverslips for 24 hours. APMS conjugated with the DNA plasmid, pCMV-DsRed-Express (Clontech Laboratories Inc., Mountain View, Calif.) were then added to cells at 37° C. for 24 hr. Cells were washed 3 times with PBS, fixed in 4% paraformaldehyde, washed 3 times in PBS, and incubated with 1 μM SYTOX Green (Molecular Probes, Invitrogen) for 5 minutes at room temperature. Coverslips were washed 3× in PBS, mounted on glass slides, and viewed using CSLM.

Intrapleural Injection (IP) of APMS:

APMS were suspended in PBS at a concentration of approximately 4×10⁷ APMS per 100 μl PBS. Samples were then mixed well and sonicated 5 times 2 sec to obtain an even suspension. C57B1/6 Mice were anaesthetized temporarily using isoflurane, and APMS were then injected into the pleural cavity between the 3^{rd} and 4^{th} right side intercostals. The needle was inserted under the ribcage so as not to puncture the lung, and 100 μl of the APMS/PBS suspension was injected. Sham control mice received 100 μl of PBS alone. Mice were observed until fully recovered and for any adverse effects, and were sacrificed after 3 or 7 days by intraperitoneal injection of sodium pentobarbital.

Intranasal Instillation (IN) of APMS:

APMS were suspended in PBS at a concentration of approximately 4×10⁷ APMS per 50 μl PBS. Samples were mixed well and sonicated 5 times 2 sec to obtain an even suspension. Mice were then anaesthetized temporarily by exposure to isoflurane in a Bell jar, and APMS were instilled by placing small drops on the nostrils of the mice and allowing them to inhale 50 μl of APMS/PBS solution. Repeated short exposures to isoflurane were necessary to complete the instillation. Mice were observed until fully recovered and for any adverse effects. Following IN administration, mice were allowed to recover for 6 or 24 hrs, after which they were euthanized as described above.

Tissue Processing:

The effects and fate of APMS in vivo were determined by examining lungs, rib cage, diaphragm, spleen and heart. Tissues were removed surgically and either placed in Tissue-Tek O.C.T.® compound (Sakura Finetek USA, Torrance, Calif.) and snap-frozen, or fixed in 4% paraformaldehyde and paraffin embedded.

Detection of APMS and Inflammatory Mediators in Pleural Lavage Fluid (PLF) or Bronchoalveolar Lavage Fluid (BALF):

Recovery of PLF: PLF was obtained following opening of the peritoneal cavity and injecting 2.0 ml-3.0 ml of PBS through the diaphragm into the chest cavity, gently massaging the chest and then recovering the fluid which was measured for volume.

Recovery of BALF: A cannula attached to a syringe was inserted into the trachea of animals, 1 ml of PBS instilled into the trachea, the lungs were gently massaged, and the fluid was then recovered and measured.

LDH and Protein Measurement: Tissue damage was measured by determining levels of LDH in fresh PLF and BALF samples using Cytotox 96 kit (Promega Corp., Madison, Wis.), and protein levels in PLF and BALF were determined using a BioRad Protein Assay Kit (Bio-Rad Laboratories, Inc., Hercules, Calif.).

Cytospins and Cell Differentials: Cytospins from PLF and BALF were made in duplicate by resuspending 50,000 cells in 450 μl of medium containing 7-10% FBS and cytocentrifugation for 10 min at 600 rpm. Slides were then air-dried and fixed for 5 min in 100% methanol, followed by Wright-Giemsa staining using HEMA 3 kit (Fisher Scientific, Kalamazoo, Mich.) as per the manufacturer's recommendations to allow identification of cell types.

CSLM and CD45 Staining for Determination of APMS Location in vivo: Tissue sections were obtained from frozen tissues, fixed in 4% paraformaldehyde and either viewed via CSLM as described above, or stained for the leukocyte common antigen, CD45. Briefly, paraformaldehyde fixed sections were rinsed in PBS, blocked for 1 hr at room temperature in 5% normal donkey serum, 0.5% bovine serum albumin (BSA) in PBS, then incubated overnight at 4° C. in a 1:500 dilution of anti-CD45 antibody (MCD4500, Caltag Laboratories, Burlingame, Calif.) in 0.5% BSA, 0.1% Triton X-100 in PBS. Slides were then rinsed in PBS and incubated with secondary antibody, donkey anti-rat conjugated to Alexa 488 (Molecular Probes), and counterstained with the nucleic acid dye, TOTO 3 (Molecular Probes, Invitrogen) or DAPI (Molecular Probes, Invitrogen). Samples were rinsed in PBS, coverslips were mounted, and slides were viewed using CSLM.

Statistical Analysis.

For in vitro experiments, at least 3 independent experiments were performed (N=2-4 per experiment). For in vivo experiments, results are representative of at least 4 mice/duplicate experiments. Statistical significance was evaluated by ANOVA using the Student Neuman-Keul's procedure for adjustment of multiple pairwise comparisons between treatment groups or using the non-parametric Kruskal-Wallis and Mann-Whitney tests. Values of p<0.05 were considered statistically significant.

Results:

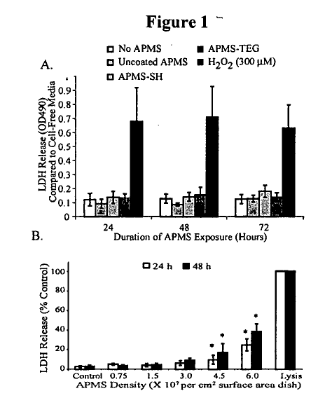

To determine the effect of surface coating on APMS uptake by C10 and MM cells, different molecules were chemically bound to the outer surface of fluorescently-tagged APMS. TEG facilitates cell uptake of APMS and TEG-APMS were not cytotoxic to lung epithelial (C10) cells as measured by release of LDH. APMS were coated with Alexa 568 and either lipid, a linker having a terminal propylamine group or a propylthiol group, or tetraethylene glycol (TEG). APMS were added at 7.5×10⁶/cm² surface area dish. To enhance the contrast, low intensity green pixels (below intensity 30) were colored black. Cell nuclei were stained with SYTOX Green. The effect of surface coatings on APMS-induced cytotoxicity was measured by the LDH assay, where 300 μM H₂O₂ was used as a positive control for LDH release (results show in FIG. 1A). A dose response study using APMS coated with TEG and measuring cell lysis by the LDH assay was performed (FIG. 1B). Complete cell lysis was used as a positive control for LDH release. Results are expressed as Mean ±SEM from 3 experiments; *=p<0.01 when compared to control group at respective time point.

When APMS were coated with lipid or a chemical linker having a terminal —NH₂ group, few APMS were observed in cells at 24 hrs. HoNwever, when APMS were coated with a linker having a terminal -SH group, increased APMS appeared to be associated with cells. Coating the APMS with TEG resulted in even further uptake by both C10 and MM cells. To determine whether surface-coated APMS particles had adverse effects on cells over time, LDH assays were performed after addition of particles for 24, 48, or 72 hrs. These studies showed that densities of APMS up to 0.75×10⁷ particles per cm² surface area of dish were nontoxic in comparison to 300 μM H₂O₂ a positive control for cell lysis (FIG. 1A). To determine the toxicity of various amounts of APMS on MM cells, APMS coated with TEG were incubated with MM cells for 24 or 48 hrs, and LDH release was measured as an indication of cell damage. As shown in FIG. 1B, APMS toxicity was not seen in MM cells until the density of APMS reached 4.5×10⁷ particles per cm² surface area dish, approximately 6-fold higher amounts than quantities used in studies below.

Since APMS coated with TEG were most effectively taken up by both cell types, the approximate time course of APMS-TEG uptake by C10 cells was then investigated in living cells using a cell membrane dye (FM4-64) and a nuclear dye (Hoechst 33342) as a counterstain. CSLM allowed the visualization of fluorescent APMS in relationship to cell membranes, as well as determination of whether APMS were inside cells or associated with the cell membrane. To clearly visualize the location of fluorescent APMS in regard to cell membranes, areas of the coverslips were visualized that had relatively few APMS present. APMS-TEG entered C10 cells within 1 hr, although both extracellular and intracellular APMS-TEG were observed at this time. At 4.5 hrs, the majority of APMS-TEG were intracellular. An orthogonal view of a CSLM image allowed the visualization of the observed microscopic field additionally in both the x,z- and y,z-planes. Fluorescent APMS were seen amongst the membrane bound cell organelles, and indicated that APMS were located in the same plane as cell nuclei, therefore they had entered the cell and were not just associated with the outer cell membrane.

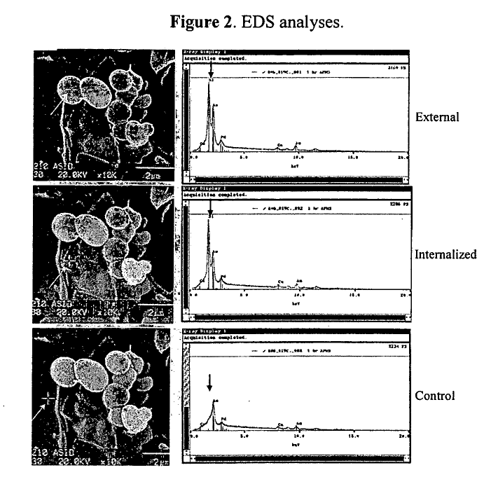

Using SEM, we determined that APMS-TEG entered MM cells as early as 30 min after their addition to medium. At all time points, most APMS were internalized, but this appeared to be a dynamic process involving interactions of the plasma membrane and microvilli with single particles or clusters of APMS-TEG, partial membrane formation around APMS at the cell surface were also noticeable. At 4 hrs, all APMS-TEG were cell-associated. SEMIEDS confirmed that spheroid particles external to the cell membrane (FIG. 2) or internalized under the plasma membrane, in contrast to areas without particles that showed no silica peaks (FIG. 2C).

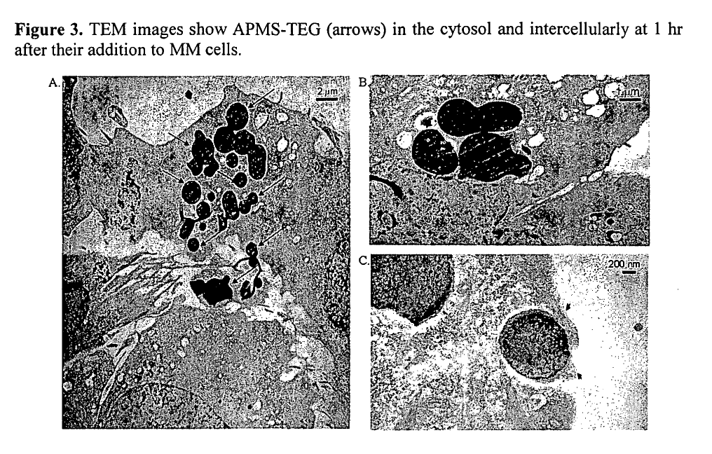

To further determine the intracellular location and lack.of cytotoxicity of APMS-TEG, MM cells were examined using TEM. At 1 hr after incubation of APMS-TEG with MM cells, particles were seen interacting with microvilli and within cells (FIG. 3A). Identification of APMS was confirmed using EDS. In all sections, intracellular APMS were not enclosed in membrane-bound phagosomes nor phagolysosomes and did not cause toxic alterations in cellular organelles (FIGS. 3B, 3C). Note the prominent microvilli interacting with intercellular beads in 3A. FIG. 3B shows perinuclear accumulation of APMS-TEG. Non-membrane bound beads surrounded by cell surface cytoplasm (arrowheads) are shown in C.

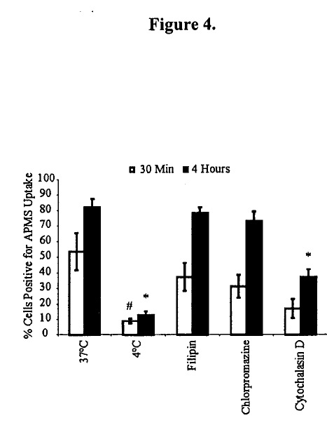

To understand the mechanisms of uptake of APMS-TEG by MM cells, flow cytometry experiments were performed using fluorescent APMS-TEG covalently linked with Alexa 488. Cell uptake of APMS is an active process not involving clathrin or caveolae-mediated mechanisms. When APMS-TEG were incubated with MM cells at 37° C., an increase in cells containing APMS occurred between 30 min and 4 hrs (FIG. 4). APMS-TEG uptake was significantly inhibited (p <0.05) when cells were maintained 4° C. We next pre-incubated cells with an inhibitor of caveolae-mediated uptake (filipin), an inhibitor of receptor-mediated endocytosis and clathrin coated pit-mediated uptake (chlorpromazine), and cytochalasin D, an agent that disrupts actin filaments, for 30 min prior to addition of APMS-TEG to cells. In comparison to the control group at 37° C., no inhibition of particle uptake was observed after pretreatment of MM cells with filipin or chlorpromazine indicating that APMS-TEG are not taken up by a mechanism involving caveolae or clathrin-coated pits (FIG. 4). However, decreased numbers of APMS-containing cells were observed after pretreatment with cytochalasin D, suggesting that APMS-TEG uptake may involve an actin-mediated process.

To determine if APMS could be used as vehicles to deliver functional plasmids, we preloaded the pores of APMS-TEG with a plasmid (pCMV-dsRedExpress) that if functionally delivered to cells, is transcribed and translated into a protein that fluoresces red. The results, showing a CSLM image of a C10 cell treated with fluorescent APMS-TEG, and counterstained with a SYTOX Green nucleic acid dye, indicated that APMS enter cells and deliver functionally expressed plasmids to cells.

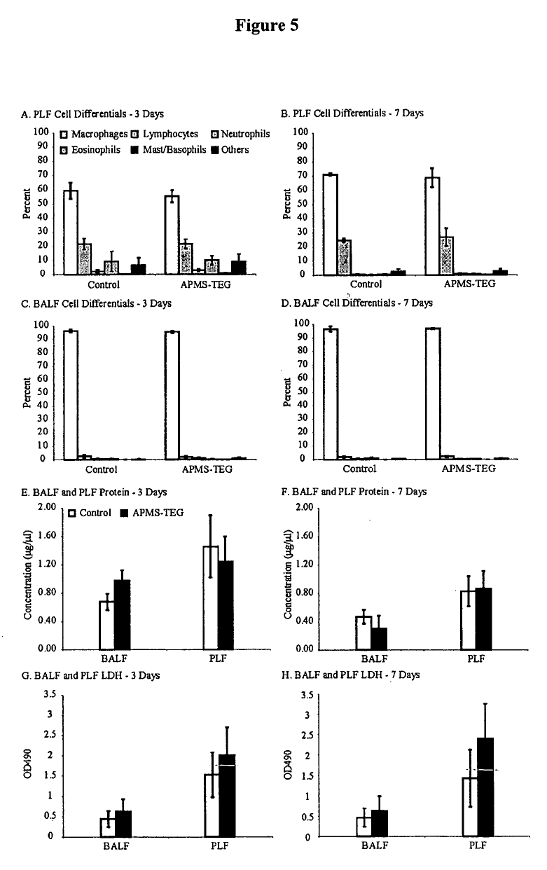

Next, we evaluated the toxicity and possible immunogenicity of APMS-TEG when injected IP or instilled IN in mice. As shown in FIG. 5A-D, injection of APMS-TEG IP did not cause a change in cell populations in PLF or BALF in comparison to sham mice injected with PBS alone. Differential cell counts, protein levels, and LDH in PLF and BALF show that APMS-TEG are not inflammatory nor toxic when injected (approximately 4×10⁷ per mouse) IP in mice. Eosinophils and basophils in control PLF fluids at 3 days in both PBS control and APMS-TEG groups may reflect acute inflammation in response to injection of PBS which has been reported in many instillation models. Moreover, injection of APMS-TEG did not alter protein or LDH levels in PLF or BALF (FIGS. 5E-H, respectively).

Finally, to determine the fate of APMS-TEG following IP injection or IN administration, CSLM was used to locate fluorescently-tagged APMS-TEG in mouse tissues. After IP injection, APMS-TEG were found in rib tissues local to the site of injection, as well as in the diaphragm, spleen and lung after 3 days. APMS-TEG were not found in the heart. In lung tissue after 3 days, APMS were located occasionally in CD45-positive leukocytes. After IN instillation, APMS-TEG were found primarily in alveolar septa of the lung where they were observed in both CD45-positive and negative cells. In all experiments, approximately 3.3×10⁷ APMS were injected IP or inhaled IN.

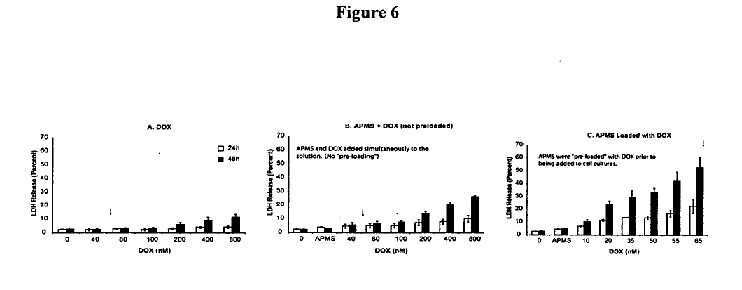

To determine whether the cell killing effects of DOX-loaded APMS were superior to addition of DOX to medium, TEG-APMS were suspended in aqueous solutions containing various amounts of DOX for 24 h. The DOX-loaded APMS were then recovered by filtration, washed briefly with H₂O, and dried in a vacuum oven before determination of DOX level. Suspensions of DOX alone or added simultaneously with non-loaded APMS at 40-800 nM (FIGS. 6A and B, respectively), and APMS loaded with DOX (10-65 nM) (FIG. 6C), were added to MM cells for 24 and 48 h before measurement of lactic dehydrogenase (LDH) release, an assay for cell lysis using H₂O₂ as a positive control. APMS delivering DOX exhibited strikingly increased toxicity to MM cells, achieving LD₅₀ levels at 65 nM DOX. APMS loaded with DOX is superior to DOX alone or added simultaneously with unloaded APMS in MM cell killing as assessed by the LDH assay.

APMS have a unique uptake mechanism and increase the amount of DOX inside cells. We have developed quantitative techniques for the detection of APMS in cells in vitro using confocal scanning laser microscopy, live cell imaging, and a flow cytometry. The latter technique, using a number of cell uptake inhibitors, has shown that the mechanisms of cellular uptake of APMS are rapid, actin-dependent, and do not involve intracellular fusion of membrane-bound APMS with lysosomes.

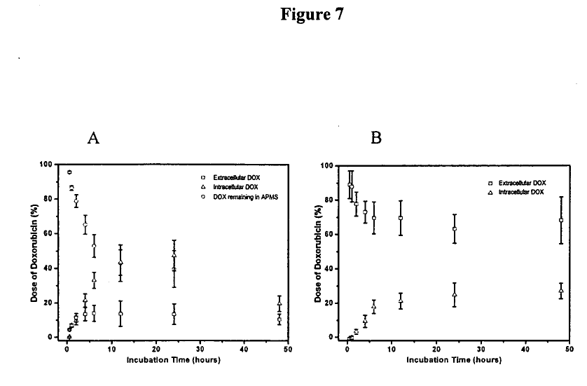

These data show the ability of APMS to circumvent cellular digestion and should permit the delivery of “cargo” that will not be degraded in phagolysosomes. We have also modified existing HPLC methods (Andersen et al, 1993, 1994; De Bruijn et al, 1999) to compare the amounts of DOX transferred to cellular interiors by DOX-loaded APMS and DOX alone. More DOX is transferred to cells when it is preloaded into APMS than when it is administered alone. Briefly, measurement of cellular DOX concentrations was accomplished by lysis and enzymatic digestion of the cellular contents, along with centrifugation. Since DOX becomes bound to DNA, we found that addition of DNase to release DOX from DNA followed by MeOH and ZnSO₄ was also necessary to insure accurate quantitation by fluorescence HPLC (since DOX is inherently fluorescent, no additional labeling was necessary). Similar methodology was used to quantify the amount of extracellular DOX, and since the total amount of DOX within the APMS particles prior to its addition to the cell culture was known, the amount of DOX remaining within the particles could be determined (FIG. 7). Intracellular and extracellular amounts of DOX were measured by fluorescence HPLC after digestion and release of DOX from cellular material (FIG. 7A). Note the sudden decrease in the amount of intracellular DOX at 48 h due to cell death and lysis. FIG. 7B illustrates a similar experiment was performed except that the DOX was added directly to the culture medium. Approximately half as much DOX was transferred to the cell in this experiment than the one shown in the left panel. At short times, a small amount (10-15%) of DQX appeared in the cell culture medium, whether through cellular lysis and diffusion out of cell membranes or through leakage from extracellular APMS. As more and more particles were taken up by cells, the amount of intracellular DOX increased until a maximum of approximately 50% of the initial amount within the particles had been released; the balance of DOX remained within the particles even at longer times. In contrast, the maximum amount of DOX transferred to the cellular interior when DOX was added directly to the culture without APMS was approximately 25% of the initial dose. It is clear that the amount of DOX transferred to the cells was at least twice as high when APMS was used. It is also important to note that the final point data point in the DOX-APMS plot (48 h) showed a sudden drop in the intracellular DOX concentration; this is because the cells had already died and had lysed their contents into the culture medium; the DOX only plot never showed this feature because an insufficient number of cells had died.

TEG-APMS enter cells of the diaphragm when administered intrathoracically (IT) and do not induce acute inflammation. TEG-APMS (˜3.3×10⁷ particles per mouse) was administered in phosphate-buffered saline (PBS) by IT injection, and CSLM was then used to determine the fate and location of APMS on frozen sections of lung, diaphragm and ribs. TEG-APMS labeled with a fluorescent dye (Alexa-568) were also used to identify intracellular particles confirmed by EDXA analysis. APMS were located in each of these tissues, the maximum uptake occurring in cells of the diaphragm at sites of injection. CSLM revealeds TEG-APMS in ribs and diaphragm 72 h after IT injection of APMS (3.3×10⁷/0.1 ml PBS) into mice. Pleural lavage fluids (PLF) and bronchoalveolar lavage fluids (BALF) from TEG-APMS injected mice and sham mice (receiving PBS alone) were evaluated for determination of leukocyte infiltration and cell/tissue damage by differential cell counts and total protein levels. In comparison to sham PBS-injected controls, no effects on these parameters occurred after-acute APMS administration (3 and 7 days). To determine the fate and clearance of APMS at these time points, the spleen, kidney and liver from these mice were examined for APMS. Briefly, tissues were digested in KOH and burned at 800° C. to remove all organic material. The remaining material was then analyzed using scanning electron microscopy (SEM) and EDXA to probe for silica). Traces of silica were found in the kidney, suggesting urinary excretion. Using CSLM, APMS were also in CD4+ cells in the spleen, suggesting transport by leukocytes to this organ. The uptake and delivery of APMS containing drugs by alveolar, pleural or peritoneal macrophages may also be advantageous as tumor macrophages are known to produce paracrine factors encouraging tumor growth and fibroblast proliferation/collagen deposition.

### REFERENCES FOR EXAMPLE 1

1. Borm, P. J., and W. Kreyling. 2004. Toxicological hazards of inhaled nanoparticles—potential implications for drug delivery. J Nanosci Nanotechnol 4(5):521-31.
2. Nel, A., T. Xia, L. Madler, and N. Li. 2006. Toxic potential of materials at the nanolevel. Science 311(5761):622-7.
3. Oberdorster, G., E. Oberdorster, and J. Oberdorster. 2005. Nanotoxicology: an emerging discipline evolving from studies of ultrafine particles. Environ Health Perspect 113(7):823-39.
4. United States Environmental Protection Agency. 2006. http://www.epa.gov/oar/particlepollution/fs20051220pm.html. Fact Sheet: Proposal to Revise the National Ambient Air Quality Standards for Particulate Matter.
5. Gallis, K. W., J. T. Araujo, K. J. Duff, J. G. Moore, and C. C. Landry. 1999. The use of mesoporous silica in chromatography. Adv Mater 11 (17):1452-1455.
6. Gallis, K. W., and C. C. Landry. 2002. Mesoporous silica and method of making same. U.S. Pat. No. 6,334,988. USA.
7. Nassivera, T., A. G. Eklund, and C. C. Landry. 2002. Size-exclusion chromatography of low-molecular-mass polymers using mesoporous silica. J Chromatogr A 973(1-2):97-101.
8. Guthrie, G. D., and B. T. Mossman, editors. 1993. Health Effects of Mineral Dusts, Reviews in Mineralogy. Mineralogic Society of America, Washington DC.
9. Solberg, S. M., and C. C. Landry. 2006. Adsorption of DNA into mesoporous silica. J Phys Chem B Condens Matter Mater Surf Interfaces Biophys 110(31):15261-8.
10. Farokhzad, O. C., J. Cheng, B. A. Teply, I. Sherifi, S. Jon, P. W. Kantoff, J. P. Richie, and R. Langer. 2006. Targeted nanoparticle-aptamer bioconjugates for cancer chemotherapy in vivo. Proc Natl Acad Sci USA 103(16):6315-20.
11. Maruyama, K. 2002. PEG-immunoliposome. Biosci Rep 22(2):251-66.
12. Olivier, J. C. 2005. Drug transport to brain with targeted nanoparticles. NeuroRx 2(1):108-19.
13. Olivier, J. C., R. Huertas, H. J. Lee, F. Calon, and W. M. Pardridge. 2002. Synthesis of pegylated immunonanoparticles. Pharm Res 19(8):1137-43.
14. Martin-Herranz, A., A. Ahmad, H. M. Evans, K. Ewert, U. Schulze, and C. R. Safinya. 2004. Surface functionalized cationic lipid-DNA complexes for gene delivery: PEGylated lamellar complexes exhibit distinct DNA-DNA interaction regimes. Biophys J 86(2): 1160-8.
15. Huang, M., W. Wu, J. Qian, D. J. Wan, X. L. Wei, and J. H. Zhu. 2005. Body distribution and in situ evading of phagocytic uptake by macrophages of long-circulating poly (ethylene glycol) cyanoacrylate-co-n-hexadecyl cyanoacrylate nanoparticles. Acta Pharmacol Sin 26(12):1512-8.
16. Sadzuka, Y., I. Sugiyama, T. Tsuruda, and T. Sonobe. 2006. Characterization and cytotoxicity of mixed polyethyleneglycol modified liposomes containing doxorubicin. Int J Pharm 312(1-2):83-9.
17. Hicke, B. J., A. W. Stephens, T. Gould, Y. F. Chang, C. K. Lynott, J. Heil, S. Borkowski, C. S. Hilger, G. Cook, S. Warren, and P. G. Schmidt. 2006. Tumor targeting by an aptamer. J Nucl Med 47(4):668-78.
18. Berna, M., D. Dalzoppo, G. Pasut, M. Manunta, L. Izzo, A. T. Jones, R. Duncan, and F. M. Veronese. 2006. Novel monodisperse PEG-dendrons as new tools for targeted drug delivery: synthesis, characterization and cellular uptake. Biomacromolecules 7(1):146-53.
19. Matsumura, Y., M. Gotoh, K. Muro, Y. Yamada, K. Shirao, Y. Shimada, M. Okuwa, S. Matsumoto, Y. Miyata, H. Ohkura, K. Chin, S. Baba, T. Yamao, A. Kannami, Y. Takamatsu, K. Ito, and K. Takahashi. 2004. Phase I and pharmacokinetic study of MCC-465, a doxorubicin (DXR) encapsulated in PEG immunoliposome, in patients with metastatic stomach cancer. Ann Oncol 15(3):517-25.
20. Greenwald, R. B., K. Yang, H. Zhao, C. D. Conover, S. Lee, and D. Filpula. 2003. Controlled release of proteins from their poly(ethylene glycol) conjugates: drug delivery systems employing 1,6-elimination. Bioconjug Chem 14(2):395-403.
21. Greenwald, R. B., Y. H. Choe, J. McGuire, and C. D. Conover. 2003. Effective drug delivery by PEGylated drug conjugates. Adv Drug Deliv Rev 55(2):217-50.
22. Greenwald, R. B., C. D. Conover, and Y. H. Choe. 2000. Poly(ethylene glycol) conjugated drugs and prodrugs: a comprehensive review. Crit Rev Ther Drug Carrier Syst 17(2):101-61.
23. Malkinson, A. M., L. D. Dwyer-Nield, P. L. Rice, and D. Dinsdale. 1997. Mouse lung epithelial cell lines—tools for the study of differentiation and the neoplastic phenotype. Toxicology 123(1-2):53-100.
24. Carpino, L. A. 1993. 1-Hydroxy-7-azabenzotriazole. An efficient peptide coupling additive. J Am Chem Soc 115(10):4397-4398.
25. Schnitzer, J. E., P. Oh, E. Pinney, and J. Allard. 1994. Filipin-sensitive caveolae-mediated transport in endothelium: reduced transcytosis, scavenger endocytosis, and capillary permeability of select macromolecules. J Cell Biol 127(5):1217-32.
26. Wang, L. H., K. G. Rothberg, and R. G. Anderson. 1993. Mis-assembly of clathrin lattices on endosomes reveals a regulatory switch for coated pit formation. J Cell Biol 123(5):1107-17.
27. Orlandi, P. A., and P. H. Fishman. 1998. Filipin-dependent inhibition of cholera toxin: evidence for toxin internalization and activation through caveolae-like domains. J Cell Biol 141(4):905-15.
28. Rejman, J., V. Oberle, I. S. Zuhorn, and D. Hoekstra. 2004. Size-dependent internalization of particles via the pathways of clathrin- and caveolae-mediated endocytosis. Biochem J 377(Pt 1): 159-69.
29. Tsakiridis, T., M. Vranic, and A. Klip. 1994. Disassembly of the actin network inhibits insulin-dependent stimulation of glucose transport and prevents recruitment of glucose transporters to the plasma membrane. J Biol Chem 269(47):29934-42.
30. Ramos-Nino, M. E., G. Vianale, T. Sabo-Attwood, L. Mutti, C. Porta, N. Heintz, and B. T. Mossman. 2005. Human mesothelioma cells exhibit tumor cell-specific differences in phosphatidylinositol 3-kinase/AKT activity that predict the efficacy of Onconase. Mol Cancer Ther 4(5):835-42.
31. Altomare, D. A., H. You, G. H. Xiao, M. E. Ramos-Nino, K. L. Skele, A. De Rienzo, S. C. Jhanwar, B. T. Mossman, A. B. Kane, and J. R. Testa. 2005. Human and mouse mesotheliomas exhibit elevated AKT/PKB activity, which can be targeted pharmacologically to inhibit tumor cell growth. Oncogene 24(40):6080-9.
32. Mossman, B. T., J. B. Kessler, B. W. Ley, and J. E. Craighead. 1977. Interaction of crocidolite asbestos with hamster respiratory mucosa in organ culture. Lab Invest 36(2):131-9.
33. Persson, H. L. 2005. Iron-dependent lysosomal destabilization initiates silica-induced apoptosis in murine macrophages. Toxicol Lett 159(2):124-33.
34. Wiewrodt, R., A. P. Thomas, L. Cipelletti, M. Christofidou-Solomidou, D. A. Weitz, S. I. Feinstein, D. Schaffer, S. M. Albelda, M. Koval, and V. R. Muzykantov. 2002. Size-dependent intracellular immunotargeting of therapeutic cargoes into endothelial cells. Blood 99(3):912-22.
35. Prabha, S., W. Z. Zhou, J. Panyam, and V. Labhasetwar. 2002. Size-dependency of nanoparticle-mediated gene transfection: studies with fractionated nanoparticles. Int J Pharm 244(1-2):105-15.
36. Desai, M. P., V. Labhasetwar, E. Walter, R. J. Levy, and G. L. Amidon. 1997. The mechanism of uptake of biodegradable microparticles in Caco-2 cells is size dependent. Pharm Res 14(11):1568-73.
37. Geiser, M., B. Rothen-Rutishauser, N. Kapp, S. Schurch, W. Kreyling, H. Schulz, M. Semmler, V. Im Hof, J. Heyder, and P. Gehr. 2005. Ultrafine particles cross cellular membranes by nonphagocytic mechanisms in lungs and in cultured cells. Environ Health Perspect 113(11):1555-60.
Abe M, Kuo W.-L., MB H., and Rosner, M: Extracellular signal-regulated kinase 7 (ERK7), a novel ERK with a c-terminal domain that regulates its activity, its cellular location, and cell growth. Mol. Cell. Biol., 19. 1301-1312, 1999
Abe J, Kusuhara M, Ulevitch R J, Berk B C, and Lee J D: Big mitogen-activated protein kinase 1 (BMK1) is a redox-sensitive kinase. J. Biol. Chem., 271: 16586-16590, 1996
Andersen A, Warren D J, Slordal L: A sensitive and simple high-performance liquid chromatographic method for the determination of doxorubicin and its metabolites in plasma. Therapeutic Drug Monitoring, 15: 455-461, 1993
Andersen A, Warren D J, Slordal L: Quantitation of cell-associated doxorubicin by high-performance liquid chromatography after enzymatic desequestration. Cancer Chemother. Pharmacol., 34: 197-202, 1994
Beck J S, Vartuli J C, Roth W J, Leonowicz M E, Kresge C T, Schmitt K D, Chu CT-W, Olson D H, Sheppard E W, McCullen S B, Higgins J B, Schlenker J L: A New Family of Mesoporous Molecular Sieves Prepared with Liquid Crystal Templates. J. Am. Chem. Soc., 114:10834, 1992
Bignon, J. (ed): Health Related Effects of Phyllosilicates. NATO ASI Series G. Ecological Sciences, Vol. G21. Berlin: Springer-Verlag, 1989.
Birner P, Ritzi M, Musahl C, Knippers R, Gerdes J, Voigtlander T, Budka H, and Hainfeliner J: A. Immunohistochemical detection of cell growth fraction in formalin-fixed and paraffin-embedded murine tissue. Am J Pathol, 158: 1991-1996, 2001
Bocchetta M, Di Resta I, Powers A, Fresco R, Tosolini A, Testa J R, Pass,H I, Rizzo P, and Carbone M: Human mesothelial cells are unusually susceptible to simian virus 40-mediated transformation and asbestos cocarcinogenicity. Proc Natl Acad Sci USA, 97: 10214-10219, 2000
Boutin C, Schiesser M, Frenay C, and Astoul P: Malignant pleural mesothelioma. Eur Respir J, 12: 972-981, 1998
Buder-Hoffmann S, Palmer C, Vacek P, Taatjes D, and Mossman B T: Different accumulation of activated extracellular signal-regulated kinases (ERK 1/2) and role in cell-cycle alterations by epidermal growth factor, hydrogen peroxide, or asbestos in pulmonary epithelial cells. Am. J. Respir. Cell. Mol. Biol., 24: 405-413, 2001
Churg A: Chrysotile, tremolite, and malignant mesothelioma in man. Chest, March; 93(3):621-8. Review, 1988
Cummins A B, Palmer C, Mossman B T, and Taatjes D J: Persistent localization of activated extracellular signal-regulated kinases (ERK1/2) is epithelial cell-specific in an inhalation model of asbestosis. Am J Pathol, 162: 713-720, 2003
Datamonitor. Global Drug Delivery: Industry Profile. November 2004. 2005 projections extrapolated.from historical market data.
Davis, G, Pfeiffer L M, and Hemenway D: Interferon production by specific lung lymphocyte phenotypes in silicosis in mice. Am J Respir. Cell Mol. Biol., 22: 491-501, 2000
De Bruijn P, Verweij J, Loos W J, Kolker H J, Planting A S T, Nooter K, Stoter G, Sparreboom A: Determination of doxorubicin and doxorubicinol in plasma of cancer patients by high-performance liquid chromatography. Analyt. Biochem., 266: 216-221, 1999
Gallis K W, Araujo J T, Duff K J, Moore J G, Landry C C: The Use of Mesoporous Silica in Liquid Chromatography. Adv. Mater., 11: 1452, 1999
Gallis K W, Eklund A G, Jull S T, Araujo J T, Moore J G, Landry C C: Using Mesoporous Silica in Liquid Chromatography. Stud. Surf. Sci. Catal., 129: 747, 2000
Gallis K W and Landry, C C: Mesoporous Silicates and Method of Making Same. U.S. Pat. No. 6,334,988, 2002
Grondin S C and Sugarbaker D J: Malignant mesothelioma of the pleural space. Oncology, 13: 919-926, 1999
Guthrie G and Mossman B T: (eds.) Health Effects of Mineral Dusts, Vol. 28, p. 1-584. Washington, DC: Mineralogical Society of America, 1993
Hartmann, M: Ordered Mesoporous Materials for Bioadsorption and Catalysis. Chem. Mater., 17: 4577, 2005
Hassan R, Bera T, Pastan I: Mesothelin: a new target for immunotherapy. Clin. Cancer Res., 10: 3937-3942, 2004
Hubler M, Sounders J E, Shade E D, Hlastala M P, Polissar N L, Glenny R W: Validation of fluorescent-labeled microspheres for measurement of relative blood flow in severely injured lungs. J Appl Physiol, 87: 2381-2385, 1999
Jänne P A: First Line Chemotherapy for Malignant Pleural Mesothelioma. In: Pass, Vogelzang, and Carbone, eds. Malignant Mesothelioma: Advances in Pathogenesis, Diagnosis, and Translational Therapies. Springer Science+Business Media, Inc., New York, N.Y., pp. 21-33, 2005
Jimenez L, Zanella C, Fung H, Janssen Y, Vacek P, Charland C, Goldberg J, and Mossman B T: Role of extracellular signal-regulated protein kinases in apoptosis by asbestos and H₂O₂. Am. J. Physiol. (Lung Cell Mol Physiol), 273: L1029-L1035, 1997
Jung M, Davis W, Taatjes D, Churg A, and Mossman B T: Asbestos and cigarette smoke cause increased DNA strand breaks and necrosis in bronchiolar epithelial cells in vivo. Free Rad. Biol. Med., 28: 1295-1299, 2000
Kamakura S, Moriguchi T, and Nishida E: Activation of the protein kinase ERK5/BMK1 by receptor tyrosine kinases. Identification and characterization of a signaling pathway to the nucleus; J Biol Chem, 274.-26563-26571, 1999
Kamp D and Mossman B T: Asbestos-associated cancers: Clinical spectrum and pathogenic mechanisms. Clin. Occup. Environ. Med., 2: 753-777, 2002
Kato Y, Kravchenko W V, Tapping R I, Han J, Ulevitch R J, and Lee J D: BMK1/ERK5 regulates serum-induced early gene expression through transcription factor MEF2C. Embo J, 16: 7054-7066, 1997
Kato Y, Tapping R I, Huang S, Watson M H, Ulevitch R J, and Lee J D: Bmk1/Erk5 is required for cell proliferation induced by epidermal growth factor. Nature, 395: 713-716, 1998
Kraus-Berthier L, Jan M, Guilbaud N, Naze M, Pierre A, and Atassi G: Histology and sensitivity to anticancer drugs of two human non-small cell lung carcinomas implanted in the pleural cavity of nude mice. Clin Cancer Res, 6: 297-304, 2000
Kresge C T, Leonowicz M E, Roth W J, Vartuli J C, Beck J S: Ordered Mesoporous Molecular Sieves Synthesized by a Liquid-Crystal Template Mechanism. Nature, 359: 710, 1992
Linden C J: Chemosensitivity spectrum of a human pleural mesothelioma xenograft grown in athymic rats. In Vivo, 5: 375-380, 1991
Mossman B T, Bignon J, Corn M, Seaton A, and Gee J: Asbestos: Scientific developments and implications for public policy. Science, 247: 294-301, 1990
Mossman B T and Churg A: State-of-the-Art: Mechanisms in the pathogenesis of asbestosis and silicosis. Am J Respir Crit Care Med, 157: 1666-1680, 1998
Mossman B T and Gee J: Asbestos related disease. N. Engl. J. Med., 320: 1721-1730, 1989
Mossman B T and Gruenert D: SV40, growth factors, and mesothelioma—another piece of the puzzle. Am. J. Respir. Cell. Mol. Biol., 26: 167-170, 2002
Mossman B T, Kamp D, and Weitzman S: Mechanisms of carcinogenesis and clinical features of asbestos-associated cancers. Cancer Invest., 14: 466-480, 1996
Mossman B T, Marsh J P, Hardwick D, Gilbert R, Hill S, Sesko A, Shatos, M, Doherty J, Weller A and Bergeron M. J: Approaches to prevention of asbestos-induced lung disease using polyethylene glycol (PEG)-conjugated catalase. Free Rad Biol Med, 2: 335-338, 1986.
Mou C-Y and Lin H-P: Control of Morphology in Synthesizing Mesoporous Silica. Pure Appl. Chem., 72: 137, 2000
Nassivera T W, Eklund A G, Landry C C: Size Exclusion Chromatography (SEC) of Low Molecular Weight Polymers Using Mesoporous Silica. J. Chromatogr. A., 973: 97, 2002
Nassivera T W and Landry C C: A New Type of Mesoporous Silicate and Method of Making Same. Provisional Patent filed, 2004
Orengo A M, Spoletini L, Procopio A, Favoni R E, De Cupis A, Ardizzoni A, Castagneto B, Ribotta M, Betta PG, Ferrini S, and Mutti L: stablishment of four new mesothelioma cell lines: characterization by ultrastructural and immunophenotypic analysis. Eur Respir J, 13: 527-534, 1999
Pass H I, Vogelzang N J, and Carbone M, eds. Malignant Mesothelioma: Advances in Pathogenesis Diagnosis and Translational Therapies. Springer Science+Business Media, Inc., New York, N.Y., pp. 21-33, 2005
Pearson, G., English, J. M., White, M. A., and Cobb, M. H. ERK5 and ERK2 cooperate to regulate NF-kappaB and cell transformation. J Biol Chem, 276: 7927-7931, 2001
Poladi RHPR and Landry C C: Synthesis, Characterization, and Catalytic Properties of MMM-1. J. Solid State Chem., 167: 363, 2002
Poladi RHPR and Landry C C: Oxidation of Octane and Cyclohexane Using a New Porous Substrate, Ti-MMM-1. Microporous Mesoporous Mater., 52: 11, 2002
Ramos-Nino M E, Scapoli L, Martinelli M, Land S, and Mossman B T: Microarray analysis and RNA silencing link fra-1 to cd44 and c-met expression in mesothelioma. Cancer Res, 63: 3539-3545, 2003
Ramos-Nino M E, Timblin C, and Mossman B T: Mesothelial cell transformation requires increased AP-1 binding activity and ERK-dependent Fra-1 expression. Cancer Res., 62: 6065-6069, 2002
Ramos-Nino M, Vianale G, Sabo-Attwood T, Mutti L, Mossman B T: Human mesothelioma cells exhibit tumor cell-specific differences in phosphatidylinositol 3-kinase/AKT activity that predict the efficacy of Onconase. Molecular Cancer Therapeutics, 4: 835-42, 2005
Robledo R, Buder-Hoffmann S, Cummins A, Walsh E, Taatjes D, and Mossman B T: Increased phosphorylated ERK immunoreactivity associated with proliferative and morphologic lung alterations following chrysotile asbestos inhalation in mice. Am. J. Pathol., 156: 1307-1316, 2000
Roggli V L, Sharma A, Butnor K J, Sporn T, Vollmer R T: Malignant mesothelioma and occupational exposure to asbestos: a clinicopathological correlation of 1445 cases. Ultrastruct Pathol, 26: 55-65, 2002
Scapoli L, Ramos-Nino M E, Martinelli M, and Mossman B T: Src-dependent ERK5 and Src/EGFR-dependent ERK1/2 activation is required for cell proliferation by asbestos. Oncogene, 23: 805-813, 2004
Schaeffer H and Weber M: Mitogen-activated protein kinases: specific messages from ubiquitous messengers. Mol. Cell. Biol., 19: 2435-2444, 1999
Smith, Aaron. Cancer Drug Boom Projected. CNNMoney [online] Sep. 19, 2005. https://money.cnn.com/2005/09/19/news/fortune500/cancerdrugs/
Standard & Poors. Industry Survey—Healthcare. Pharmaceuticals. Jul. 21, 2005.
Sterman D H, Kaiser L R, and Albelda S M: Advances in the treatment of malignant pleural mesothelioma. Chest, 116. 504-520, 1999
Stober E, Fink A, Bohn E: Controlled Growth of Monodisperse Silica Spheres in the Micron Size Range. J. Coll. Interface Sci. 26: 62, 1968
Sugarbaker D J, Flores R M, Jaklitsch M T, Richards W G, Strauss G M, Corson J M, DeCamp Jr. M M, Swanson S J, Bueno R, Lukanich J M, Baldini E H, and Mentzer S J: Resection margins, extrapleural nodal status, and cell type determine postoperative long-term survival in trimodality therapy of malignant pleural mesothelioma: results in 183 patients. J Thorac Cardiovasc Surg, 117: 54-63, 1999
US Dept Health & Human Services: Mortality in Libby, Montana. Agency for Toxic Substances and Disease Registry Health Consultation, Vol. (1979-1998), 2002
Veronese F M, Schiavon O, Pasut G, Mendichi R, Andersson L, Tsirk A, Ford J, Wu G, Kneller S, Davies J, and Duncan, R: PEG-Doxorubicin Conjugates: Influence of Polymer Structure on Drug Release, in Vitro Cytotoxicity, Biodistribution, and Antitumor Activity. Bioconjugate Chem., 16: 775-784, 2005
World Health Organization: Silica, some silicates, coal dust and para-aramid fibrils. IARC Monographs on the Evaluation of Carcinogenic Risks of Humans, Vol. 68, 1996.
Zanella C, Posada J, Tritton T, and Mossman B: Asbestos causes stimulation of the ERK-1 mitogen-activated protein kinase cascade after phosphorylation of the epidermal growth factor receptor. Cancer Res., 56: 5334-5338, 1996
Zanella C, Timblin C, Cummins A, Jung M, Goldberg J, Raabe R, Tritton, T, and Mossman B T: Asbestos-induced phosphorylation of epidermal growth factor receptor is linked to c-fos expression and apoptosis. Am. J. Physiol. (Lung Cell Mol Physiol), 277: L684-L693, 1999

### Example 2

### Modification and Optimization of APMS for Delivery and Uptake of shRNA Constructs

In this example the shRNA constructs were used to inhibit the ERK1/2 and ERK5 pathways that we have shown are upregulated in MM cells and participate in tumor cell growth and chemoresistance. Although it has been demonstrated that lung-specific siRNA delivery can be achieved by nasal instillation without the need for viral vectors and produces acute effects in mediating lung injury (Zhang et al., 2004), we expect that APMS will be more a effective vehicle for uptake and delivery of shRNA constructs into the thoracic cavity.

ERK1 and 2 are the major members of the mammalian mitogen-activated protein kinase (MAPK) family. ERK1/2 pathway is activated in rat mesothelial cells in response to asbestos, the carcinogen responsible for the induction of greater than 80% of human MMs (Zanella et al., 1996). It has since been shown that both the ERK1/2 and ERK5 pathways contribute to cell proliferation by asbestos (Scapoli et al., 2004) Recent work in human MM cells confirms that these pathways are important in MM cell proliferation as well as chemoresistance. Moreover, we have developed and validated shmek5 constructs.

A purpose of these studies is to validate in vitro that APMS loaded with shRNA constructs both increases the preclinical effectiveness of the constructs and improves the inhibition of cell signaling pathways in human MM cells.

Extracellular Signal-Regulated Kinases (ERKs) in MM.

Several survival pathways have been identified in MMs by the Mossman laboratory and by others. These include the Extracellular Signal-Regulated Kinases (ERKs), Nuclear Factor-kB (NF-kB), and phosphatidylinositol 3-kinase (PI3K)/AKT pathways. The ERK group of Mitogen-Activated Protein Kinases (MAPKs) includes the widely studied mammalian enzymes, ERK1 and ERK2, and other more recently discovered family members such as ERK7, which has constitutive activity in serum-starved cells (Abe, et al., 1999). ERKs are characterized by the dual phosphorylation motif, Thr-Glu-Tyr. The mammalian ERK1 and ERK2 MAPKs are activated by signaling pathways that are initiated often by stimulation of cell surface receptors, a common point of integration being the activation of the small G protein, Ras. Phosphorylated ERKs translocate to the nucleus and phosphorylate transcription factors such as Elk-1 that are essential to transcriptional activation of c-fos, a component of the AP-1 transcription factor. In a recent analysis of 50 biopsies of MMs, increased phosphorylated (activated) ERK1/2 expression was significantly increased and translocated to nuclei. This observation is not surprising, as the ERK1/2 pathway is triggered by a multiplicity of growth factors (EGF, HGF, PDGF, IGF-1, etc.) that are produced in an autocrine fashion by MMs (Mossman and Gruenert, 2002). ERK5 also has been linked to c-jun expression as well as to EGFR-mediated cell proliferation and cell cycle progression in several cell types (Abe et al., 1996; Kato et al,., 1997; Kato et al., 1998; Kamakura et al., 1999). We have shown herein that the ERK5 pathway is linked to chemoresistance of MM cells The fact that ERK1/2 and ERK5 may act cooperatively in cell proliferation/survival and transformation (Scapoli et al., 2004; Pearson, et al., 2001) suggests that both of these pathways may act to increase cell survival of MMs after chemotherapy.

Current si/shRNA Delivery Methods.

Delivering si/shRNAs in vivo is in its infancy but involves viral vectors or nonviral approaches such as those using synthetic polymers. Viral vectors are potentially limiting in terms of clinical use because of their cell-type specificity and potentially adverse effects in patients including inflammatory potential. Although a limited number of publications describe systemic delivery of siRNAs with synthetic or viral vectors in vivo, a recent report demonstrates effective intranasal heme oxygenase-1 (HO-1) siRNA delivery without a vector or transfection agent for prevention of acute lung epithelial cell apoptosis after ischemia-reperfusion injury (Zhang et al., 2004).

TEG-APMS Effectively Transfers DNA Plasm ids to Epithelial and Mesothelioma Cells In Vitro.

To demonstrate whether TEG-APMS can deliver drugs or constructs such as nucleic acid plasmids to cells, a plasmid encoding red fluorescent protein (RFP) was loaded into the pores of APMS and added to C10 cells for 24 hr. Cells were fixed and examined by CSLM which showed cell delivery of the plasmid. The fact that red fluorescence was not seen in surrounding individual cells verified that the plasmid did not dissociate from the APMS in aqueous medium.

Quantification of Uptake of Linear and Plasmid DNA by APMS.

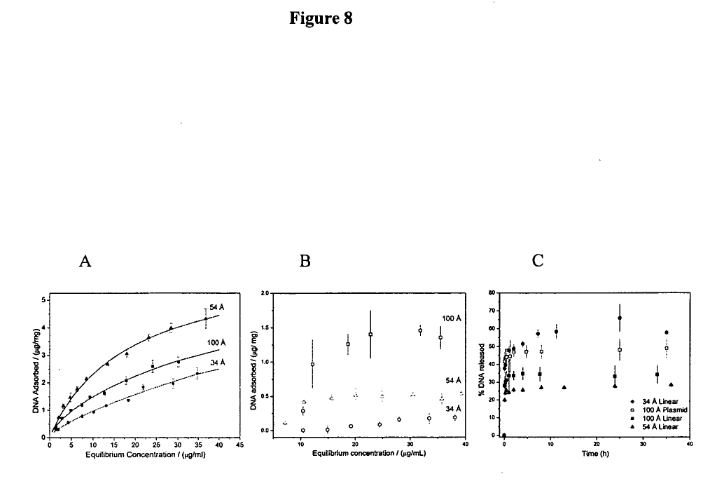

Linear and plasmid DNA sequences were adsorbed into the pores of spherically shaped acid-prepared mesoporous silica (APMS). DNA adsorption of a simple 760 bp sequence into the interior of the mesoporous material was confirmed using confocal microscopy of sequences containing fluorescently labeled-DNA molecules and by porosity measurements. Based on these results, the extent of adsorption was measured at various concentrations using UV-Visible spectrophotometry to establish adsorption isotherms (FIGS. 8A, 8B). Since the amount of DNA adsorbed was far in excess of the amount that would be adsorbed onto the external surface, it was clear that the DNA is being adsorbed into the internal pore volume of the particles. APMS alone adsorbed a negligible amount of DNA; however, exchanging divalent cations such as Mg⁺² into the pores of APMS prior to DNA uptake was found to cause a significant amount of DNA to be adsorbed. DNA adsorption was also dependent on the pore diameter of APMS. Materials with pores 54 or 100 Å in diameter absorbed substantially more linear DNA per unit of surface area than material with 34 Å pores. The amount of linear DNA adsorbed could also be significantly increased by using aminopropyltriethoxysilane to covalently link ammonium ions to the surface. Post-synthetic modification of the silica surface with aminopropyl groups increased the maximum DNA adsorption to 15.7 ag/mg silica, for materials with pore diameters of 100 Å. This indicates that DNA binds more strongly in the presence of the ammonium group compared to the metal counter-ions. Calculation and comparison of Freundlich and Langmuir constants for these adsorption processes indicated that intermolecular interactions between the DNA molecules within the pores are significant when the effective pore diameter is small, including materials with larger pores that were modified with organosilane. In contrast, a somewhat lower uptake of plasmid DNA was observed under the same conditions, and a larger pore diameter was required to show effective uptake of plasmid due to the fact that the plasmid DNA cannot distort as extensively as linear DNA to fit into the pores (FIG. 8C). However, APMS with a 100 Å pore diameter loaded with plasmid DNA released a similar percentage of its DNA as compared to materials loaded with linear DNA, showing that for materials with sufficient pore diameters, uptake and release of larger DNA constructs is not problematic.

Development of shRNA Constructs for ERK5.

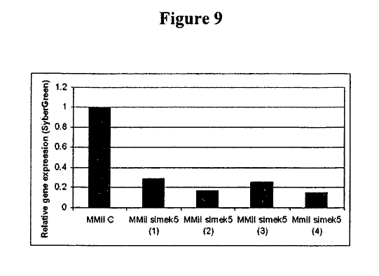

Because a validated shERK1 (MAPK1) expression plasmid is commercially available for suppression of human ERK1/2, we have focused on developing si/sh constructs for ERK5 targeting the upstream MEK5. For transient transfection experiments we used short interfering RNAs, or siRNA. Briefly, RNA oligonucleotides were purchased from either Dharmacon or Ambion, annealed in vitro, and transfected into cells using either Oligofectamine (Invitrogen) or Transit-TM (Mirus). Ambion and Dharmacon guarantee their siRNAs will knock-down expression of specific gene products by 75% within 48 hr. Immunofluorescence microscopy of cells transfected with siRNAs to lamin A/C was used to develop transfection conditions. In our experience, once satisfactory transfection conditions are established, expression of target mRNAs in cells transfected with siRNA is inhibited about 70-90% for 1-5 days. The timing and magnitude of the decrease in protein expression depends on the stability of the protein target. However, initial reduction of expression of very stable proteins such as pRB takes 1-3 days. Our more recent approach for long term RNAi expression involved the use of short hairpin RNA (shRNA) expression constructs because siRNAs cannot be used to establish stable cell lines and shRNA will allow more stable delivery and effects in vivo. While there are a wide variety of plasmids and viruses for expressing interfering shRNAs in cells, we have used the pSilencer plasmids from Ambion for generating stable cell lines. These plasmids express shRNAs from polIII promoters (e.g. U6 or histone HI), which are then processed by Dicer before entering the RTSC complex. As for siRNAs, the efficacy of shRNAs is dependent on the target sequence, stability of the target protein, and the nature of the gene product (e.g. one cannot make a shRNA stable cell line against Cdc6 or Cdc45 because these factors are essential for cell division). We have tested different si/shRNA constructs to down-regulate the expression of erk5. The efficiency of knockdown (62%) was reduced to 47% using the shRNA construct based on the sequence selected. Western blotting and RT-QPCR (Taqman) were used to examine expression of ERK5 protein and mRNA in MM1 cells 72 hr after transfection with siRNA to erk5. siRNA to scrambled target sequences were used as control. mRNA levels in stable cell lines expressing shRNA to erk5 were examined by PCR or RT-QPCR. Scrambled target sequences to erk5 were used as control. Qther siRNA constructs to knockdown the erk5 upstream regulator mek5 have proven more efficient as shown in data from four new constructs evaluated recently (FIG. 9). Knockdown of mek5 in MMil cell line with 4 siRNA constructs. QRT-PCR using SyberGreen. Gene expression was compared to the scramble control group. A stable cell line has been created using this construct.

### REFERENCES FOR EXAMPLE 2

Abe M, Kuo W.-L., MB H., and Rosner, M: Extracellular signal-regulated kinase 7 (ERK7), a novel ERK with a c-terminal domain that regulates its activity, its cellular location, and cell growth. Mol. Cell. Biol., 19. 1301-1312, 1999
Abe J, Kusuhara M, Ulevitch R J, Berk B C, and Lee J D: Big mitogen-activated protein kinase 1 (BMK1) is a redox-sensitive kinase. J Biol Chem, 271: 16586-16590, 1996
Beck J S, Vartuli J C, Roth W J, Leonowicz M E, Kresge C T, Schmitt K D, Chu CT-W, Olson D H, Sheppard E W, McCullen S B, Higgins J B, Schlenker J L: A new family of mesoporous molecular sieves prepared with liquid crystal templates. J. Am. Chem. Soc. 114:10834, 1992
Buder-Hoffmann S, Palmer C, Vacek P, Taatjes D, and Mossman B T: Different accumulation of activated extracellular signal-regulated kinases (ERK 1/2) and role in cell-cycle alterations by epidermal growth factor, hydrogen peroxide, or asbestos in pulmonary epithelial cells. Am. J. Respir. Cell. Mol. Biol., 24. 405-413, 2001
Churg A: Chrysotile, tremolite, and malignant mesothelioma in man. Chest, March; 93(3):621-8. Review, 1988
Cummins A B, Palmer C, Mossman B T, and Taatjes D J: Persistent localization of activated extracellular signal-regulated kinases (ERK1/2) is epithelial cell-specific in an inhalation model of asbestosis. Am J Pathol, 162: 713-720, 2003
Fox-Tucker J: The cancer market outlook to 2010. Business Insights 2005
Gallis K W, Araujo J T, Duff K J, Moore J Q, Landry C C: The use of mesoporous silica in liquid chromatography. Adv. Mater. 11:1452, 1999
Gallis K W, Eklund A G, Jull S T, Araujo J T, Moore J G, Landry C C: Using mesoporous silica in liquid chromatography. Stud. Surf. Sci. Catal. 129:747, 2000
Gallis K W and Landry, C C: Mesoporous silicates and method of making same. U.S. Pat. No. 6,334,988, 2002
Grondin S C and Sugarbaker D J: Malignant mesothelioma of the pleural space. Oncology, 13: 919-926, 1999
Guthrie G and Mossman B T: (eds.) Health effects of mineral dusts, Vol. 28, p. 1-584. Washington, DC: Mineralogical Society of America, 1993
Hannon G, Rossi J: Unlocking the potential of the human genome with RNA interference. Nature 431: 371-278, 2004
Hassan R, Bera T, and Pastan I: Mesothelin: a new target for immunotherapy. Clin. Cancer Res. 10: 3937-3942, 2004
Ho W C, Dickson K M, and Barker P A: Nuclear factor-kB induced by doxorubicin is deficient in phosphorylation and acetylation and represses nuclear factor-kB dependent transcription in cancer cells. Cancer.Res. 65: 4273-4280, 2005
Howard K: Unlocking the money-making potential of RNAi. Nature Biochemistry, 21:1441-1446, 2003
Jänne P A: First Line Chemotherapy for Mailgnant Pleural Mesothelioma. In: Pass, Vogelzang, and Carbone, eds. Malignant mesothelioma: advances in pathogenesis, diagnosis, and translational therapies. Springer Science+Business Media, Inc., New York, N.Y., pp. 21-33, 2005
Jimenez L, Zanella C, Fung H, Janssen Y, Vacek P, Charland C, Goldberg J, and Mossman B T: Role of extracellular signal-regulated protein kinases in apoptosis by asbestos and H₂O₂. Am. J. Physiol. (Lung Cell Mol Physiol), 273: L1029-L1035, 1997
Jung M A Davis W P, Taayes D J, Churg A, and Mossman B T: Asbestos and cigarette smoke cause increased DNA strand breaks and necrosis in bronchiolar epithelial cells in vivo. Free Rad. Biol. Med. 28:1295-1299, 2000
Kamakura S, Moriguchi T, and Nishida E: Activation of the protein kinase ERK5/BMK1 by receptor tyrosine kinases. Identification and characterization of a signaling pathway to the nucleus; J Biol Chem, 274: 26563-26571, 1999
Kamp D and Mossman B T: Asbestos-associated cancers: Clinical spectrum and pathogenic mechanisms. Clin. Occup. Environ. Med., 2: 753-777, 2002
Kato Y, Kravchenko V V, Tapping R I, Han J, Ulevitch R J, and Lee J D: BMK1/ERK5 regulates serum-induced early gene expression through transcription factor MEF2C. Embo J, 16: 7054-7066, 1997
Kato Y, Tapping R I, Huang S, Watson M H, Ulevitch R J, and Lee J D: Bmk1/Erk5 is required for cell proliferation induced by epidermal growth factor. Nature, 395: 713-716, 1998
Kresge C T, Leonowicz M E, Roth W J, Vartuli J C, Beck J S: Ordered Mesoporous Molecular Sieves Synthesized by a Liquid-Crystal Template Mechanism. Nature 359:710, 1992
Mossman B T and Churg A: State-of-the-Art: Mechanisms in the pathogenesis of asbestosis and silicosis. Am J Respir Crit Care Med, 157:1666-1680, 1998
Mossman B T and Gee J: Asbestos related disease. N. EngI. J. Med., 320: 1721-1730, 1989
Mossman B T and Gruenert D: SV40, growth factors, and mesothelioma—another piece of the puzzle. Am. J. Respir. Cell. Mol. Biol., 26: 167-170, 2002
Mossman B T, Kamp D, and Weitzman S: Mechanisms of carcinogenesis and clinical features of asbestos-associated cancers. Cancer Invest. 14: 466-480, 1996
Nassivera T W, Eklund A G, Landry C C: Size exclusion chromatography (SEC) of low molecular weight polymers using mesoporous silica. J. Chromatogr. A. 973:97, 2002
Nassivera T W and Landry C C: A new type of mesoporous silicate and method of making same. Provisional Patent filed, 2004
Ordonez N G: The immunohistochemical diagnosis of mesothelioma: a comparative study of epithelial mesothelioma and lung adenocarcinoma. Am. J. Surg. Path. 27: 1031-1051, 2003
Pass H I, Vogelzang N J, and Carbone M, eds. Malignant mesothelioma: advances in pathogenesis diagnosis and translational therapies. Springer Science+Business Media, Inc., New York, N.Y., pp. 21-33, 2005
Pearson, G., English, J. M., White, M. A., and Cobb, M. H. ERK5 and ERK2 cooperate to regulate NF-kappaB and cell transformation. J Biol Chem, 276: 7927-7931, 2001
Ramos-Nino M E, Scapoli L, Martinelli M, Land S, and Mossman B T: Microarray analysis and RNA silencing link fra-1 to cd44 and c-met expression in mesothelioma. Cancer Res, 63. 3539-3545, 2003
Ramos-Nino M E, Timblin C, and Mossman B T: Mesothelial cell transformation requires increased AP-1 binding activity and ERK-dependent Fra-1 expression. Cancer Res., 62: 6065-6069, 2002
Ramos-Nino M E and Mossman B T: Microarray analysis and RNA silencing to determine genes functionally important in mesothelioma. In: Appasani, K., ed. RNA interference technology: from basic science to drug development, Cambridge University Press, Cambridge, UK, 447-469, 2005
Robinson B W, Creaney J, Lake R, Nowak A, Musk A W, de Klerk N, Winzell P, Hellstron K E, and Hellstrom I: Mesothelin-family proteins and diagnosis of mesothelioma. Lancet 362: 1612-1616, 2003
Robinson B W and Lake R A: Medical progress: malignant mesothelioma. New Engl.J. Med. 353: 1591-1603, 2005
Robledo R, Buder-Hoffmann S, Cummins A, Walsh E, Taatjes D, and Mossman B T: Increased phosphorylated ERK immunoreactivity associated with proliferative and morphologic lung alterations following chrysotile asbestos inhalation in mice. Am. J. Pathol., 156: 1307-1316, 2000
Roggli V L, Sharma A, Butnor K J, Sporn T, Vollmer R T: Malignant mesothelioma and occupational exposure to asbestos: a clinicopathological correlation of 1445 cases. Ultrastruct Pathol, March-April; 26(2):55-65, 2002
Scapoli L, Ramos-Nino M E, Martinelli M, and Mossman B T: Src-dependent ERK5 and Src/EGFR-dependent ERK1/2 activation is required for cell proliferation by asbestos. Oncogene, 23: 805-813, 2004
Schaeffer H and Weber M: Mitogen-activated protein kinases: specific messages from ubiquitous messengers. Mol. Cell. Biol., 19: 2435-2444, 1999
Solberg S M and Landry C C: Adsorption of DNA into mesopprous silica. J. Phys. Chem. B, submitted, 2006.
Sterman D H and Albelda S M: Advances in the diagnosis, evaluation, and management of malignant pleural mesothelioma. Respirology 10: 266-283, 2005
Stober E, Fink A, Bohn E: Controlled Growth of Monodisperse Silica Spheres in the Micron Size Range. J. Coll. Interface Sci. 26:62, 1968
Sugarbaker D J, Flores R M, Jaklitsch M T, Richards W G, Strauss G M, Corson J M, DeCamp Jr. M M, Swanson S J, Bueno R, Lukanich J M, Baldini E H, and Mentzer S J: Resection margins, extrapleural nodal status, and cell type determine postoperative long-term survival in trimodality therapy of malignant pleural mesothelioma: results in 183 patients. J Thorac Cardiovasc Surg, 117: 54-63, 1999
US Dept Health & Human Services: Mortality in Libby, Montana. Agency for Toxic Substances and Disease Registry Health Consultation, Vol. (1979-1998), 2002
Veronese F M, Schiavon O, Pasut G, Mendichi R, Andersson L, Tsirk A, Ford J, Wu G, Kneller S, Davies J, and Duncan, R: PEG-doxorubicin conjugates: influence of polymer structure on drug release, in Vitro Cytotoxicity, Biodistribution, and Antitumor Activity. Bioconjugate Chem. 16: 775-784, 2005
Zanella C, Posada J, Tritton T, and Mossman B: Asbestos causes stimulation pf the ERK-1 mitogen-activated protein kinase cascade after phosphorylation of the epidermal growth factor receptor. Cancer Res., 56: 5334-5338, 1996
Zanella C, Timblin C, Cummins A, Jung M, Goldberg J, Raabe R, Tritton T, and Mossman B T: Asbestos-induced phosphorylation of epidermal growth factor receptor is linked to c-fos expression and apoptosis. Am. J. Physiol. (Lung Cell Mol Physiol), 277: L684-L693, 1999
Zhang X, Shan P, Jiang D, Noble P W, Abraham N G, Kappas A, and Lee P T: Small interfering RNA targeting heme oxygenase-1 enhances ischemia-reperfusion-induced lung apoptosis. J. Biol. Chem. 279: 10677-10684, 2004

### Example 3

### Acid-Prepared Mesoporous Spheres (APMS) Increase Efficacy of Doxorubicin Toxicity in Human Mesothelioma Cells

Materials and Methods:

Cells and Reagents. Three human mesothelioma cell lines (MO, M26, M27, kind gifts from Dr. Luciano Mutti, Maugeri Foundation, Pavia, Italy, and Dr. Michele Carbone, University of Hawaii Cancer Center, Honolulu, Hawaii) were incubated at 37° C. and 5% CO₂ and grown to approximately 80-90% confluent in complete medium, consisting of DMEM/F12 50/50 (10-092-CV, Mediatech, Inc. Herndon, Va.), and supplemented with 10% fetal bovine serum (35-010-CV, Mediatech), 0.1 μg/ml hydrocortisone (H-0135, Sigma, St. Louis, Mo.), 2.5 μg/ml insulin, 2.5 μg/ml transferrin, 2.5 ng/ml sodium selenite (1-1884, Sigma) and penicillin-streptomycin (50 U/ml penicillin G, 50 μg/ml streptomycin sulfate) (15140-122, GIBCO, Carlsbad, Calif.). Unless otherwise specified, other reagents were purchased from Sigma (St. Louis, Mo.).

Synthesis of APMS. Cetyltrimethylammonium bromide (CTAB) (4.9 mM) was suspended in 53.75 ml of an ethanol and distilled water mixture (at a 1:2.8 ratio). Concentrated hydrochloric acid (HCl, 3.7 ml) was added, and the mixture stirred until fully dissolved. Tetraethoxysilane (TEOS, 4.29 ml) was then added with stirring for 5 min, followed by addition of 4.67 ml of 0.5 M sodium fluoride (NaF, in water), the mixture heated at 373 K for 40 min, and the resulting white precipitate captured by filtration, washed 1× with distilled H₂O, and air dried. The synthesized APMS were placed under high vacuum to remove the remaining moisture.

Synthesis of APMS-TEG and Fluorescently Labeled APMS. To modify their surface, APMS were reacted with an organosilane containing the target functionality. As an example, to synthesize secondary amine-terminated particles, APMS (1 g) and N-methyl-propylaminetrimethoxysilane (2 mM) were added to hexane (20 ml), and the mixture was heated at 333 K for 2 h. After cooling to room temperature, the solid was filtered, washed with hexane and ethanol, and dried at 373 K in air. To remove the surfactant (CTAB), 1 g of amine-modified APMS with N-methyl-propylamine functionality (APMS-NHMe) was refluxed twice for 6 h in ethanol followed by extensive washes with ethanol. The resulting APMS-NHMe then was dried under high vacuum. A half gram of APMS-NHMe was suspended in 10 ml of anhydrous ethanol containing mono-tosylated tetraethylene glycol (Ts-TEG) (0.5 mM). The mixture was then refluxed for 6 h. The resulting APMS-TEG was captured by filtration, washed with ethanol, and dried in air. The APMS-TEG were placed under high vacuum to remove the remaining solvent. APMS were covalently labeled with appropriate fluorescent dyes, such as Alexa-488 succinimide ester, by forming peptide bonds.

Preloading APMS with Doxorubicin (DOX). APMS coated with TEG were preloaded with DOX by incubating them in DOX-HCl (D-1515, Sigma) for 24 h, followed by complete dehydration in a vacuum. The concentration of DOX within the APMS were calculated theoretically and verified via HPLC following hydration overnight in water or methanol at room temperature, as previously described (13). Concentrations of DOX in cells were measured. We have also modified existing HPLC methods (Andersen A, Warren D J, Slordal L: A sensitive and simple high-performance liquid chromatographic method for the determination of doxorubicin and its metabolites in plasma. Therapeutic Drug Monitoring, 15: 455-461, 1993; Andersen A, Warren D J, Slordal L: Quantitation of cell-associated doxorubicin by high-perfornance liquid chromatography after enzymatic desequestration. Cancer Chemother. Pharmacol., 34: 197-202, 1994; De Bruijn P, Verweij J, Loos W J, Kolker H J, Planting A S T, Nooter K, Stoter G, Sparreboom A: Determination of doxorubicin and doxorubicinol in plasma of cancer patients by high-performance liquid chromatography. Analyt. Biochem., 266: 216-221, 1999) to compare the amounts of DOX transferred to cellular interiors by DOX-loaded APMS and DOX alone. Measurement of cellular DOX concentrations was accomplished by lysis and enzymatic digestion of the cellular contents, along with centrifugation. Since DOX becomes bound to DNA, we found that addition of DNase to release DOX from DNA followed by MeOH and ZnSO₄ was also necessary to insure accurate quantitation by fluorescence HPLC (since DOX is inherently fluorescent, no additional labeling was necessary). Similar methodology was used to quantify the amount of extracellular DOX, and since the total amount of DOX within the APMS particles prior to its addition to the cell culture was known, the amount of DOX remaining within the particles could be determined.

Treatment of Cells with APMS. Cells were plated and grown to 70-80% confluence at 37° C. in complete medium. Medium was aspirated and replaced with maintenance medium containing 0.5% FBS, and incubated for 24 h. APMS were then resuspended in medium containing 0.5% FBS at a concentration of approximately 6×10⁷ APMS per 100 μl, mixed well, and sonicated 5× for 2 sec, to disperse any clumps. Fifty μl were then added to cells at a final concentration of 7.5×10⁶/cm² surface area dish (i.e., ˜185 particles/cell).

Sample Preparation for Scanning Electron Microscopy (SEM). Cells were grown on Thermanox plastic coverslips (Nalge Nunc International, Naperville, Ill.) in 12-well plates as described above, and treated with APMS for various amounts of time. Coverslips were washed 2× for 5 min with 0.1 M Millonig's phosphate buffer (pH 7.2), then fixed in 1:1 H₂O dilution of Kamovsky's fixative (2.5% glutaraldehyde, 1% paraformaldehyde) at 4° C. for 45 min. Samples were then washed with Millonig's phosphate buffer (pH 7.2), and post-fixed in osmium tetroxide (OsO₄) at 4° C. for 30-45 min. Samples were then dehydrated in graded ethanols, from 35% to 100%.

Scanning Electron Microscopy (SEM). Samples prepared as described above were critical point dried using liquid CO₂ as the transition fluid in a Samdri PVT-3B critical point dryer (Tousimis Research Corporation, Rockville, Md.). Specimens were mounted on aluminum specimen stubs using conductive graphite paint and allowed to dry, and were sputter-coated for 4-5 min with gold and palladium in a Polaron sputter coater (Model 5100). Specimens were then examined with a JSM 6060 scanning electron microscope (JEOL USA, Inc., Peabody, Mass.).

Cell Viability Assays. Cell lysis was measured by determining levels of LDH in the medium of treated samples using Cytotox 96 kit (Promega Corporation, Madison, Wis.) as per the manufacturer's recommendations. Briefly, cells were treated with APMS and/or DOX alone and in combination for varying amounts of time, and 50 μl of the supernatant was removed (in triplicate) and placed into wells of a 96-well plate. Fifty μl of substrate buffer was added to each well, and the plate was incubated for 30 min at room temperature in the dark. Fifty μl of stop solution was then added, and plates were read at 490 nm.

Cell viability was measured in treated cells by using the calorimetric MTS Assay, CellTiter 96 AQueous One Solution Cell Proliferation Assay (Promega) as per the manufacturer's recommendations. Briefly, 5×10³ cells were plated in a 96-well microtiter plate and allowed to recover overnight. Medium was aspirated, and cells were maintained in a low serum-containing medium for 24 h prior to treatment for varying amounts of time with APMS and/or DOX. MTS reagent was added, plates were incubated at 37° C. for 2-3 h and the absorbance of the wells was read at 492 nm as a measure of cell viability.

Cells were plated in 12-well plates and allowed to recover overnight. Medium was aspirated, and cells were maintained in a low serum medium for 24 h at 37° C. Cells were then treated with APMS and/or DOX for varying amounts of time and were viewed by phase contrast microscopy for morphology using an Olympus IX70 inverted microscope (Olympus America Inc., Lake Success, N.Y.).

Intrapleural Injection (IP) of APMS. Fluorescently-tagged APMS were suspended in PBS at a concentration of approximately 4×10⁷ APMS per 100 μl PBS. Samples were then mixed well and sonicated 5×2 sec to obtain an even suspension. C57B1/6 Mice were anaesthetized temporarily using isoflurane, and APMS were then injected into the pleural cavity between the 3^{rd} and 4^{th} right side intercostals. The needle was inserted under the ribcage so as not to puncture the lung, and 100 μl of the APMS/PBS suspension was injected. Sham control mice received 100 μl of PBS alone. Mice were observed until full recovery and for any adverse effects, and were sacrificed after 3 or 7 days by intraperitoneal injection of sodium pentobarbital.

Tissue Processing. The effects and fate of APMS in vivo were determined by examining lungs, rib cage, diaphragm, spleen and heart. Tissues were removed surgically and either placed in Tissue-Tek O.C.T.® compound and snap-frozen, or fixed in 4% paraformaldehyde and paraffin embedded.

Confocal Scanning Laser Microscopy (CSLM) and CD45 Staining for Determination of APMS Location in vivo. Tissue sections were obtained from frozen tissues, fixed in 4% paraformaldehyde and either viewed via CSLM, or stained for the leukocyte common antigen, CD45. Briefly, paraformaldehyde fixed sections were rinsed in PBS, blocked for 1 h at room temperature in 5% normal donkey serum, 0.5% bovine serum albumin (BSA) in PBS, then incubated overnight at 4° C. in a 1:500 dilution of anti-CD45 antibody (MCD4500, Caltag Laboratories, Burlingame, Calif.) in 0.5% BSA, 0.1% Triton X-100 in PBS. Slides were then rinsed in PBS and incubated with secondary antibody, donkey anti-rat conjugated to Alexa488, and counterstained with the nucleic acid dye, TOTO 3 (Molecular Probes, Invitrogen) or 4′,6-diamidino-2-phenylindole (DAPI) (Molecular Probes, Invitrogen). Samples were rinsed in PBS, coverslips were mounted, and slides were viewed on a Zeiss LSM 510 META confocal scanning laser microscope (Carl Zeiss Microimaging, Thornwood, N.Y.).

Mechanisms of Cytotoxicity. To explore mechanisms of toxicity of DOX at low concentrations delivered by APMS, levels of phosphorylated histone protein H2AX (γH2AX), a marker of DNA double strand breaks, and Apoptosis Inducing Factor (AIF), a marker of mitochondrial mediated apoptosis, were analyzed.

Immunocytochemical Analysis ofphosphorylated histone protein H2AX (γH2AX). Monolayers of cells were grown on glass coverslips and treated as described above. Cells were then washed in PBS, then fixed in 4% paraformaldehyde and permeabilized in 0.1% Triton X-100. Cells were blocked for 1 h at room temperature in PBS containing 3% bovine serum albumin (BSA), then incubated in a 1:500 dilution of mouse monoclonal anti-phospho-histone H2AX antibody (#05-636, Upstate Cell Signaling, Charlottesville, Va.) in 3% BSA overnight at 4° C. Cells were then washed in PBS and incubated in a 1:300 dilution of goat anti-mouse IgO conjugated to Alexa-647 fluorescent tag (Molecular Probes, Invitrogen) in PBS for 45 min at room temperature in the dark. Cells were washed and counterstained with DAPI nucleic acid stain (Molecular Probes, Invitrogen), at a 1:1000 dilution in PBS for 5 min at room temperature. Coverslips were then washed and mounted on glass slides using Aqua Poly/Mount (#18606, Polysciences, Inc., Warrington, Pa.) and analyzed using a Zeiss LSM 510 META confocal scanning laser microscope (Carl Zeiss Microimaging).

Immunocytochemical Analysis of Apoptosis Inducing Factor (AIF). Monolayers of cells were grown on glass coverslips and treated as described above. Cells were washed in PBS/0.1% Tween 20 (PBST) and fixed in 4% paraformaldehyde, permeabilized in 0.1% Triton X-100, then blocked for 1 h at room temperature in PBS containing 5% BSA and 2% normal goat serum (blocking buffer). Samples were incubated in a 1:500 dilution of rabbit monoclonal anti-AIF antibody (#1020-1, Epitomics Inc., Burlingame, Calif.) in blocking buffer overnight at 4° C. Cells were then washed in PBST and incubated in a 1:300 dilution of goat anti-rabbit Ig5 conjugated to Alexa-647 fluorescent tag (Molecular Probes, Invitrogen) in PBS for 45 min at room temperature in the dark. Coverslips were washed and counterstained with DAPI nucleic acid stain (Molecular Probes, Invitrogen), at a 1:1000 dilution in PBS for 5 min at room temperature, washed 2× in PBS, and slides coverslipped using Aqua Poly/Mount (#18606, Polysciences, Inc.). Cells were then analyzed, using a Zeiss LSM 510 META confocal scanning laser microscope (Carl Zeiss Microimaging).

Western Blot Analyses for Phosphorylated ERK's1/2 and AKT To determine whether DOX at low concentrations delivered by APMS modified signaling pathways associated with cell survival, nearly confluent MM cells were washed 3× with cold PBS, scraped from culture plates, and collected by centrifugation at 14,000 rpm for 1 min. The pellet was resuspended in lysis buffer [20 mM Tris (pH 7.4), 1% Triton X-100, 10% glycerol, 137 mM NaCl, 2 mM EDTA, 25 mM β-glycerophosphate, 1 mM Na₃VO₄, 2 mM pyrophosphate, 1 mM PMSF, 10 μg/ml leupeptin, 1 mM DTT, 10 mM NaF, 1% aprotinin], incubated at 4° C. for 15 min, and centrifuged at 14,000 rpm for 20 min. Protein concentrations were determined using a Bio-Rad RC-DC Assay (Bio-Rad Laboratories, Hercules, Calif.). Twenty pg of protein in sample buffer [62.5 mM Tris-HCl (pH 6.8), 2% sodium dodecyl sulfate (SDS), 10% glycerol, 50 mM dithiothreitol, 0.1% w/v bromophenol blue] was resolved by electrophoresis in 10% SDS-polyacrylamide gels, and transferred to nitrocellulose using a Mini Trans-Blot Cell apparatus (Bio-Rad Laboratories). Blots were incubated in blocking buffer [Tris-buffered saline (TBS) containing 5% nonfat dry milk, 0.1% Tween-20] for 1 h, washed 3× for 5 min each in TBS/0.1% Tween-20, and incubated at 4° C. overnight with antibodies specific to phospho-AKT or total AKT, phospho-ERK1/2 or total ERK1/2 all at a 1:1000 dilution (Cell Signaling Technology, Beverly, Mass.). Blots were then washed 3× with TBS/0.1% Tween-20 and incubated with a specific peroxidase-conjugated secondary antibody at a dilution of 1:3,000 (Amersham Pharmacia Biotech, Piscataway, N.J.) for 1 h. Blots were washed 3× in TBS/0.1% Tween-20, and protein bands were visualized with the ECL Western Blotting Detection Reagents (Amersham Biosciences, GE Healthecare UK Limited, Buckinghamshire, UK) and quantitated using a Bio-Rad phosphorimager.

NF-κB Electrophoretic Mobility Shift Assay. Electrophoretic gel mobility shift assays were used to assess the binding of NF-κB to DNA. Nuclear extracts of MM cells treated with 80 nM DOX, 100 μM DOX, APMS-TEG, APMS-TEG preloaded with 80 nM DOX (APMS-DOX80) for 4 h, were prepared as described previously (14). Samples were treated with or without TNF-α, 1 h prior to harvesting nuclear protein. The amount of protein in each sample was determined with the Bio-Rad protein assay (Bio-Rad). A ³²P-end-labeled double stranded oligodeoxynucleotide representing the specific element that contains an NF-κB consensus sequence (E3292, Promega, Madison, Wis.) was incubated with 3 μg of extract as described previously (15). The components of the NF-κB complex were identified by antibodies specific for p50 and p65 proteins (Santa Cruz Biotechnology, Inc., Santa Cruz, Calif.). Autoradiograms were developed and quantitated with a Bio-Rad phosphorimager.

Statistical Analysis. For in vitro experiments, at least 3 independent experiments were performed (N=2-4 per experiment). For in vivo experiments, results are representative of at least 4 mice/duplicate experiments. Statistical significance was evaluated by ANOVA using the Student Neuman-Keul's procedure for adjustment of multiple pairwise comparisons between treatment groups or using the non-parametric Kruskal-Wallis and Mann-Whitney tests. Values of P<0.05 were considered statistically significant.

Results:

APMS-TEG are taken up by MM cells in vitro and into adjacent tissues following intrapleural injection. We have shown previously that APMS-TEG are taken up into MM cells via actin-mediated mechanisms (13). In studies here, MM cells actively engulf particles using a phagocytic-like mechanism. When APMS-TEG were incubated with MM cells for 2 h, using SEM, APMS were seen associated with MM cell membranes, either as individual APMS or in clusters. Additionally, after 4 h, APMS were seen at the cell membrane interacting with pseudpod-like projections from the MM cells, and some APMS were seen under the cell membranes.

To determine whether APMS-TEG could enter and remain in tissues local to the site of injection, C57BL/6 mice were injected intrapleurally with 3.3×10⁷ fluorescent APMS-TEG. Mice were sacrificed after three days, and tissues, both adjacent to, and distal to the pleura were harvested. Using CSLM, APMS were found local to the site of injection, in rib tissue, lung, and diaphragm. Other tissues were also examined to determine the fate of the APMS post-injection. APMS were seen occasionally in CD45-positive leukocytes of the spleen, but have not been seen in the heart or kidney.

APMS delivery increased DQX cytotoxicity in human MM cells in vitro. To determine the effect of APMS-TEG on DOX-mediated cell viability, cytotoxicity dose-response curves of human mesQthelioma cells were performed. Varying concentrations of DOX were incubated with MM cells for 24 or 48 h, and LDH release into the medium was determined as a measure of cell damage. At the doses tested, little toxicity of DOX was seen, with doses of DOX ranging from 800 nM to 2000 nM resulting in 10-13% cell death after 48 h, compared to total LDH released from lysed cells.

To see if delivery of DOX by APMS could affect DOX efficacy, pores of APMS-TEG were preloaded with varying amounts of DOX, and then incubated with MM cells. Using APMS as vehicles to carry DOX into cells led to a dramatic increase in cell toxicity, compared to the efficacy of DOX alone. APMS preloaded to provide a final concentration of 20 nM DOX provides a similar level of LDH release as does 2000 nM DOX alone. Further, the cytotoxic effect seen when cells are treated with APMS preloaded with DOX is dose related, with the lethal dose to kill 50% of MM cells in 48 h being approximately 65 nM, which is significantly lower than doses of DOX needed to cause any toxicity in these cells when treated with drug alone.

When APMS-TEG were simultaneously incubated with MM cells along with the varying doses of DOX, only a slight increase in toxicity was seen compared to DOX alone, but to a much lower extent than if cells were treated with APMS preloaded with DOX. In this case, similar toxicity was seen after 48 h in cells treated with 200 nM DOX simultaneously with APMS, compared to cells treated with higher doses (1-2 μM) of DOX alone. When cells are treated with higher doses of DOX simultaneously with APMS, a dose-related increase in toxicity was seen, but an LD50 is not achieved with the DOX doses studied. This indicates that APMS preloaded with DOX dramatically increases the efficacy of DOX, compared to DOX alone or simultaneously incubating MM cells with DOX and APMS.

To further verify the effect of APMS preloaded with DOX on the viability of MM cells, we incubated APMS preloaded to give a final concentration of 80 nM (APMS-DOX) with MM cells for varying amounts of time, and assessed cell viability using an MTS assay. When cells were treated with either 80 nM DOX, APMS alone or were untreated, there was no significant difference seen in cell viability. However, when cells were treated with APMS-DOX, over time, there was a significant decrease in cell viability compared to controls, further confirming the LDH results.

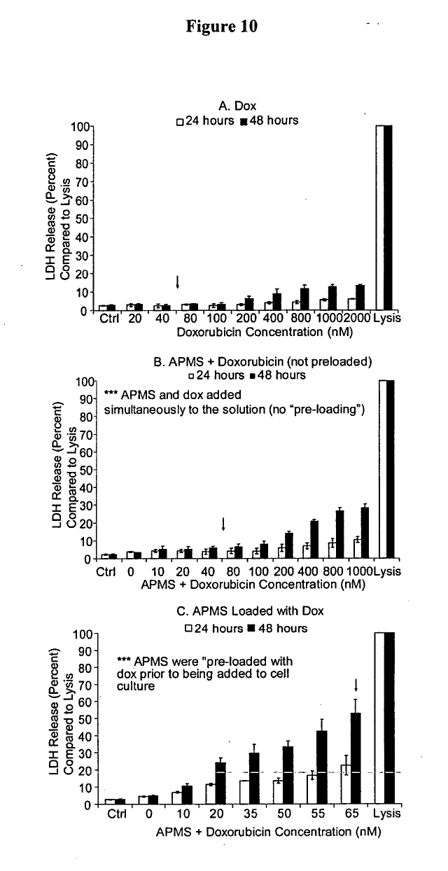

APMS-DOX effect was seen in multiple human MM cell lines. To confirm that the delivery of DOX by APMS increases the efficacy of DOX, we compared the effect of APSM-DOX treatment in three different MM cell lines, MO, M26, and M27. Phase contrast microscope images were taken of cells exposed to water (control), 80 nM DOX, 160 nM DOX, 7.5×10⁶/cm² surface area dish APMS-TEG, 15×10⁶/cm² surface area dish APMS-TEG, APMS-DOX (80 nM), or APMS-DOX (160 nM) (FIG. 10A), and LDH release was measured to compare cytotoxicities (FIG. 10B-D). The amounts of APMS-TEG that were added equals the amount of APMS-DOX added to achieve the two different DOX concentrations. Images indicate that in each cell line, APMS-DOX has the greatest impact on cell morphology. Of the three cell lines, MO cells were most sensitive to APMS-DOX, showing dramatically increased susceptibility to APMS-DOX (80 nM) after 24, 48 and 72 h, compared to cells treated with 80 nM DOX alone or APMS-TEG. M26 cells also showed increased LDH release in cell treated with APMS-DOX compared to either APMS alone or DOX alone. Similar results were seen in M27 cells, where again, cells treated with APMS-DOX exhibited increased LDH release and adverse effects on cell morphology. Together, these results emphasize that using APMS as vehicles to deliver DOX dramatically increases the efficacy of DOX compared to administering the drug alone. Because the phenomenon of APMS-DOX increasing the efficacy of DOX in all cell lines, and the greatest effect of APMS-DOX treatment on cytotoxicity was seen in MO cells, we continued the remainder of these studies using APMS-DOX (80 nM) in MO cells.

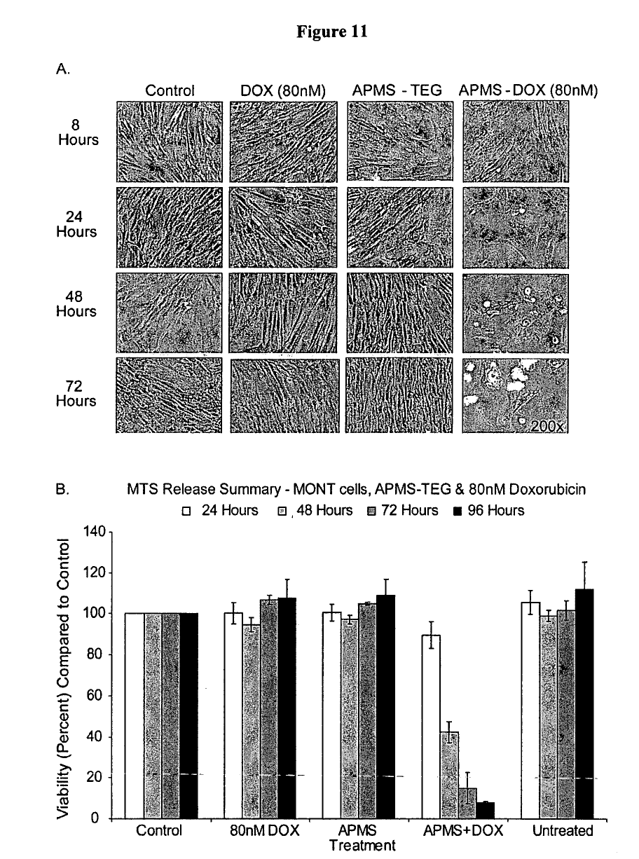

APMS-DOX treatment did not increase phosphorylation of histone protein H2AX (γH2AH). Because the mechanism of DOX action in cells includes DNA damage and double strand breaks (16, 17), immunocytochemical assays were performed to determine levels of γH2AX, a marker of DNA strand breaks (FIG. 11) (18). The numbers of cells counted that were positive for the presence of γH2AX is shown in FIG. 11A. A representative CSLM image (FIG. 11B), shows that levels of γH2AX are increased after 8 h in cells exposed to both 80 nM DOX and in cells treated with APMS-DOX80 compared to either control treated cells or cells exposed to APMS-TEG alone. The data show that gH2AX levels are increased in MM cells exposed to both DOX and APMS-DOX, to the same extent, at both 8 and 24 h. This indicates that even though 80 nM DOX is cytotoxic only when delivered in APMS, MM cells exposed to 80 nM DOX alone as well as APMS-DOX exhibit phosphorylated histone protein H2AX. It is also important to note the APMS-TEG alone did not cause any increase in γH2AX.

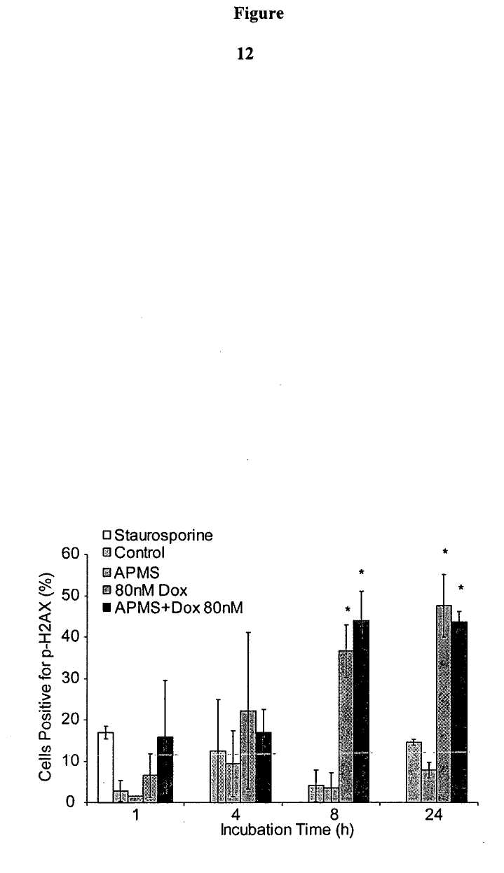

APMS-DOX treatment did not increase nuclear localization of apoptosis inducing factor (AIF). Qne of the outcomes of treatment of cells with DOX is apoptosis (16). AIF is a protein that is predominantly located in the mitochondria of resting cells, and upon cell damage can translocate to the nucleus where its effects are pro-apQptotic (19, 20). AIF was analyzed following treatment of cells with DOX and APMS (FIG. 12). The percent of cells exhibiting nuclear localized AIF indicated that the presence of APMS-TEG and APMS-DOX did not cause a significant increase in the number of cells containing nuclear AIF after 24 h exposure. CSLM images verified that nuclear localization of AIF was not significantly different in cells treated with APMS-DOX, compared to untreated control cells after 24 h.

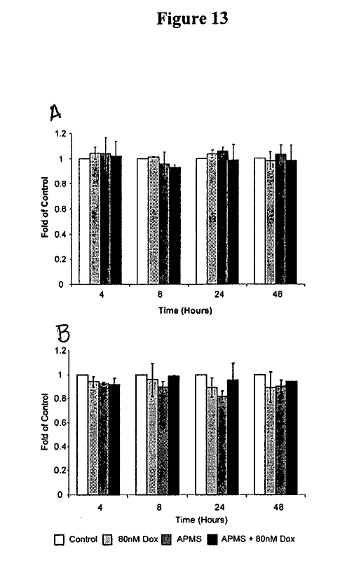

ERK1/2 and AKT phosphorylation were not affected by APMS-DOX treatment. Several proteins have been implicated as survival proteins in mesotheliomas and other tumors. Included are ERK1/2 and AKT (21, 22). To determine if APMS-DOX works through a mechanism that involves ERK1/2 or AKT, APMS-DOX cytotoxicity was investigated by incubating APMS with MM Cells for varying amounts of time, and performing Western blot analysis to determine levels of phosphorylated ERK1/2 and AKT. The chart shown in FIG. 13 is a summary of at least two independent Western Blot experiments, indicating the ratio of phosphorylated to total ERK1/2, as determined by densitometry analysis. At 4, 8, 24 and 48 h, 80 nM DOX, APMS-TEG and APMS-DOX do not significantly affect the phospho-ERK1/2: total ERK1/2 ratio. Similarly for AKT, at each time point, 80 nM DOX, APMS-TEG and APMS-DOX did not significantly affect the phospho-AKT: total AKT ratios.

APMS-DOX may work through an NF-κB-mediated mechanism. Another signaling pathway implicated in cell survival in many cancer cells is the NF-κB pathway (23). To determine if APSM-DOX exerts its cytotoxic affects by modulating NF-κB activity, we examined the effect of APMS-DOX on NF-κB-to-DNA binding. Using an electrophoretic mobility shift assay (EMSA), NF-κB-DNA binding activity was determined in response to 4 h exposure to APMS-TEG, 100 μM DOX (lethal dose), 80 riM DOX, and APMS-DOX, or 1 h exposure to TNF-α (a known inducer of NF-κB activity). When MM cells were incubated with APMS-TEG, there was no increase in DNA binding of the NF-κB subunits p50, p65 or the dimeric NF-κB complex (p65/p50). A 1 hr incubation with TNF-α dramatically increased DNA binding. When cells were incubated with 100 μM DOX (a lethal concentration), NF-κB-DNA binding activity was ablated, and this could not be reversed by adding TNF-α for 1 h prior to harvesting the cells. Compared to controls, 80 nM DOX increased the NF-κB-DNA binding, and the addition of TNF-α 1 hr prior to harvesting the cells further increased the DNA binding. When APMS-DOX we incubated with MM cells for 4 h, NF-κB-DNA binding activity was inhibited compared to controls, and this inhibition could not be reversed by the addition of TNF-α for 1 hr prior to harvesting the cells, a result similar to that seen with the lethal exposure to DOX (100 μM).

### REFERENCES FOR EXAMPLE 3

1. Mossman B T, Gee J B. Asbestos-related diseases. N Engl J Med 1989;320:1721-30.
2. Robinson B W, Lake R A. Advances in malignant mesothelioma. N Engl J Med 2005;353:1591-603.
3. Carbone M, Kratzke R A, Testa J R. The pathogenesis of mesothelioma. Semin Oncol 2002;29:2-17.
4. Vogelzang N J. Emerging insights into the biology and therapy of malignant mesothelioma. Semin Oncol 2002;29:35-42.
5. Powers A, Carbone M. The role of environmental carcinogens, viruses and genetic predisposition in the pathogenesis of mesothelioma. Cancer Biol Ther 2002;1:348-53.
6. Pisick E, Salgia R. Molecular biology of malignant mesothelioma: a review. Hematol Oncol Clin North Am 2005;19:997-1023, v.
7. Pass H, Carbone M, Vogelzang N, eds. Malignant Mesothelioma: Advances in Pathogenesis, Diagnosis, and Translational Therapies, Springer Science+Business Media, Inc., 2005.
8. Sterman D H, Albelda S M. Advances in the diagnosis, evaluation, and management of malignant pleural mesothelioma. Respirology 2005;10:266-83.
9. Grondin S C, Sugarbaker D J. Pleuropneumonectomy in the treatment of malignant pleural mesothelioma. Chest 1999;116:450S-4S.
10. Krug L M. An overview of chemotherapy for mesothelioma. Hematol Oncol Clin North Am 2005;19:1117-36, vii.
11. Tomek S, Emri S, Krejcy K, Manegold C. Chemotherapy for malignant pleural mesothelioma: past results and recent developments. Br J Cancer 2003;88:167-74.
12. Gallis K, Landry C Mesoporous silicates and method of making same. U.S., 2002.
13. Blumen S R, Cheng K, Ramos-Nino M E, et al. Unique Uptake of Acid-Prepared Mesoporous Spheres by Lung Epithelial and Mesothelioma Cells. Am J Respir Cell Mol Biol 2006; in press.
14. de Bruijn P, Verweij J, Loos W J, et al. Determination of doxorubicin and doxorubicinol in plasma of cancer patients by high-performance liquid chromatography. Anal Biochem 1999;266:216-21.
15. Timblin C, BeruBe K, Churg A, et al. Ambient particulate matter causes activation of the c-jun kinase/stress-activated protein kinase cascade and DNA synthesis in lung epithelial cells. Cancer Res 1998;58:4543-7.
16. Shukla A, Timblin C, BeruBe K, et al. Inhaled particulate matter causes expression of nuclear factor (NF)-kappaB-related genes and oxidant-dependent NF-kappaB activation in vitro. Am J Respir Cell Mol Biol 2000;23:182-7.
17. Swift L P, Rephaeli A, Nudelman A, Phillips D R, Cutts S M. Doxorubicin-DNA adducts induce a non-topoisomerase II-mediated form of cell death. Cancer Res 2006;66:4863-71.
18. Burden D A, Osheroff N. Mechanism of action of eukaryotic topoisomerase II and drugs targeted to the enzyme. Biochim Biophys Acta 1998;1400:139-54.
19. Huang X, Halicka H D, Traganos F, Tanaka T, Kurose A, Darzynkiewicz Z. Cytometric assessment of DNA damage in relation to cell cycle phase and apoptosis. Cell Prolif 2005;38:223-43.
20. Cheung E C, Joza N, Steenaart N A, et al. Dissociating the dual roles of apoptbsis-inducing factor in maintaining mitochondrial structure and apoptosis. Embo J 2006;25:4061-73.
21. Oh Y K, Shin K S, Kang S J. AIF translocates to the nucleus in the spinal motor neurons in a mouse model of ALS. Neurosci Lett 2006;406:205-10.
22. Altomare D A, You H, Xiao G H, et al. Human and mouse mesotheliomas exhibit elevated AKT/PKB activity, which can be targeted pharmacologically to inhibit tumor cell growth. Oncogene 2005;24:6080-9.
23. Scapoli L, Ramos-Nino M E, Martinelli M, Mossman B T. Src-dependent ERK5 and Src/EGFR-dependent ERK1/2 activation is required for cell proliferation by asbestos. Oncogene 2004;23:805-13.
24. Andriollo M, Favier A, Guiraud P. Adriamycin activates NF-kappaB in human lung carcinoma cells by IkappaBalpha degradation. Arch Biochem Biophys 2003;413:75-82.
25. Zalipsky S. Functionalized poly(ethylene glycol) for preparation of biologically relevant conjugates. Bioconjug Chem 1995;6:150-65.
26. Vinu A, Hossain K Z, Ariga K. Recent advances in functionalization of mesoporous silica. J Nanosci Nanotechnol 2005;5:347-71.
27. Kresge C, Leonowicz M, Roth W, Vartuli J, Beck J. Ordered mesoporous molecular sieves synthesized by a liquid-crystal template mechanism. Nature 1992;359:710-2.
28. Beck J, Vartuli J, Roth W, et al. A new family of mesoporous molecular sieves prepared with liquid crystal templates. J Am Chem Soc 1992;114:10834-43.
29. Ling Y H, Priebe W, Perez-Soler R. Apoptosis induced by anthracycline antibiotics in P388 parent and multidrug-resistant cells. Cancer Res 1993;53:1845-52.
30. Tokarska-Schlattner M, Zaugg M, Zuppinger C, Wallimann T, Schlattner U. New insights into doxorubicin-induced cardiotoxicity: the critical role of cellular energetics. J Mol Cell Cardiol 2006;41:389-405.
31. Minotti G, Menna P, Salvatorelli E, Cairo G, Gianni L. Anthracyclines: molecular advances and pharmacologic developments in antitumor activity and cardiotoxicity. Pharmacol Rev 2004;56:185-229.
32. Lu P. Monitoring cardiac function in patients receiving doxorubicin. Semin Nucl Med 2005;35:197-201.
33. Ramos-Nino M E, Vianale G, Sabo-Attwood T, et al. Human mesothelioma cells exhibit tumor cell-specific differences in phosphatidylinositol 3-kinase/AKT activity that predict the efficacy of Onconase. Mol Cancer Ther 2005;4:835-42.
34. Ho W C, Dickson K M, Barker P A. Nuclear factor-kappaB induced by doxorubicin is deficient in phosphorylation and acetylation and represses nuclear factor-kappaB-dependent transcription in cancer cells. Cancer Res 2005;65:4273-81.
35. de Melo M, Gerbase M W, Curran J, Pache J C. Phosphorylated extracellular signal-regulated kinases are significantly increased in malignant mesothelioma. J Histochem Cytochem 2006;54:855-61.

### Example 4

### Preparation of APMS-sCD4

Amino propyltriethoxysilane (4.68 mL, 20.0 mmol) and Fmoc-O-succinimide (6.88 g, 20.4 mmol) were dissolved in dichloromethane, and stirred at room temperature for 2 hours to yield 7.8 g (90% yield) of (3-triethoxysilylpropyl)-carbamic acid 9H-fluorenylmethyl ester (Fmoc-APTES) after purification by flash chromatography.

FmocAPTES (2.22 g, 0.50 equivalents based on SiO₂) and APMS (0.60 g) reacted for 24 to 48 hours in anhydrous (% water=0) toluene at 90-100° C. providing APMS-NHFmoc. (yield cannot be determined in this manner; it depends on the pore diameter, surface area, etc. since APMS is a solid) The amount of linker loaded onto the surface as 0.75 mmol/g as determined by thermogravimetric analysis.

APMS-NHFmoc was selectively deprotected by treatment with 5% piperidine in DMF at room temperature for 20 minutes.

The free amine moiety on the external particle surface selectively reacted with either 3-(p-nitro-pyridyldithio)propionic acid or 11-(p-nitro-2-pyridyldithio)undecanoic acid in the presence of HOBt (3 equivalents based on the amount of free amine, as determined by UV/Visible spectroscopy), diisopropylcarbodiimide (3 equivalents), catalytic DMAP (3 equivalents) in DMF. The reaction was complete in 24-48 hours. Acylation of the free amine on the internal pore surface was achieved by treatment with acetic anhydride (1.5 equivalents based on UV/Visible spectroscopy) in the presence of triethylamine (2.5 equivalents) in refluxing dichloromethane for 3 hours. Functionalization with the fluorophore was accomplished reacting the product of the above steps with Cys-CD4-Alexa Fluor₅₆₈ in a mixture of phosphate buffered saline and acetonitrile (2:1 v/v) at pH 6-7. The reaction mixture was stirred for 6-12 hours at room temperature to provide APMS surface-modified with fluorescently labeled CD4 protein. Deprotection of the Fmoc on the internal surfaces by treatment with 5% piperidine in DMF at room temperature for 24-48 hours provided the free amine on the internal pore surface, which was further functionalized with Alexa Fluor₄₈₈. This reaction was achieved by coupling Alex Fluor₄₈₈ succinimidyl ester (excess equivalents) to the free amine in the presence of NaHCO₃ (1 mL, 0.1 M) at room temperature. In 2 hours the desired bi-functionalized product was obtained.

The foregoing written specification is considered to be sufficient to enable one skilled in the art to practice the invention. The present invention is not to be limited in scope by examples provided, since the examples are intended as a single illustration of one aspect of the invention and other flnctionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for delivering a biological agent to a tissue, comprising
administering to a tissue of a subject a mesoporous inorganic oxide spherical particle coated with a polyethylene glycol group, wherein the mesoporous inorganic oxide spherical particle has pores loaded with a biological agent in an effective amount to deliver the biological agent to the tissue.

2. The method of claim 1, wherein the polyethylene glycol group is tetraethylene glycol (TEG).

3. The method of claim 1, wherein the mesoporous inorganic oxide spherical particle has a linker attached to an external surface.

4. The method of claim 3, wherein the linker is —Cₙ—SSpyNO₂, wherein n is 1-20.

5. The method of claim 3, wherein the linker is attached to a protein.

6. The method of claim 5, wherein the protein is a glycoprotein.

7. The method of claim 6, wherein the glycoprotein is CD4 or a fragment thereof.

8. The method of claim 5, wherein the protein is an antibody.

9. The method of claim 5, wherein the protein is a pleural tissue binding protein.

10. The method of claim 5, wherein the protein is a lung tissue binding protein.

11. The method of claim 5, wherein the protein is an epithelial cell binding protein.

12. The method of claim 3, wherein the linker is attached to a tumor molecule binding agent.

13. The method of claim 1, wherein the biological agent is a chemotherapeutic.

14. The method of claim 13, wherein the chemotherapeutic is doxorubicin, carmustine, cisplatin, dacarbazine, LY294002, or PX866.

15. The method of claim 1, wherein the biological agent is an anti-viral agent.

16. The method of claim 15, wherein the anti-viral agent is an anti-HIV agent.

17.-19. (canceled)

20. The method of claim 1, wherein the pores have a pore volume of greater than 0.75 cm³/g and an average pore diameter of greater than 37 Angstroms.

21. The method of claim 1, wherein the pores have a pore volume of greater than 1.0 cm³/g and an average pore diameter of greater than 50 Angstroms.

22. The method of claim 1, wherein the pores have a pore volume of greater than 1.3 cm³/g.

23. The method of claim 1, wherein the pores have an average pore diameter of between 2 and 200 nm.

24. The method of claim 1, wherein the mesoporous inorganic oxide spherical particle has a particle size between 0.5 and 10 μm.

25. The method of claim 1, wherein the subject has melanoma.

26. A method for delivering a biological agent to a tissue, comprising administering to a tissue of a subject a mesoporous inorganic oxide spherical particle having one or more pores with a pore volume of greater than 0.75 cm³/g and an average pore diameter of greater than 37 Angstroms, wherein the pores are loaded with a biological agent in an effective amount to deliver the biological agent to the tissue.

27.-49. (canceled)

50. A method of treating lung or pleural disease in a subject comprising:
administering to a subject in need thereof a mesoporous inorganic oxide spherical particle coated with a polyethylene glycol group, wherein the mesoporous inorganic oxide spherical particle has pores loaded with a lung or pleural therapeutic agent in an effective amount to treat the disease.

51.-73. (canceled)

74. A composition comprising:
a mesoporous inorganic oxide spherical particle coated with a polyethylene glycol group, wherein the mesoporous inorganic oxide spherical particle has pores loaded with a biological agent.

75.-94. (canceled)

95. A composition, comprising
a mesoporous inorganic oxide spherical particle having one or more pores with a pore volume of greater than 0.75 cm³/g and an average pore diameter of greater than 37 Angstroms, wherein the pores are loaded with a biological agent.

96.-114. (canceled)

115. A composition, comprising
a mesoporous inorganic oxide spherical particle having one or more pores, wherein the pores are loaded with a chemotherapeutic agent and wherein a linker is attached on one end to an external surface of the mesoporous inorganic oxide spherical particle and on another end to a tumor molecule binding agent.

116.-129. (canceled)
